(19) United States
(12) Patent Application Publication
(10) Pub. No.: US 2026/0256409 A1
(43) Pub. Date: Sep. 3, 2026

(54) VARIABLE-SIZE SEGMENTATION OF AUDITORY EVOKED POTENTIALS FOR COGNITIVE IMPAIRMENT DETECTION

(71) Applicant: Neso Technology Inc., Newark, DE (US)

(72) Inventor: Navid Banafshe, Dusseldorf (DE)

(21) Appl. No.: 19/554,385

(22) Filed: Mar. 2, 2026

Related U.S. Application Data
(60) Provisional application No. 63/766,051, filed on Mar. 3, 2025.

(51) Int. Cl. A61B 5/38; A61B 5/291

(52) U.S. Cl. CPC A61B 5/38; A61B 5/291

(57) ABSTRACT

A system for segmenting auditory-evoked potentials (AEPs) from electroencephalography (EEG) signals comprises an auditory stimulator configured to present controlled auditory stimuli to a subject, an EEG acquisition device including a plurality of sensors adapted to detect electrical brain signals from a scalp of the subject in response to the auditory stimuli, and a data processing unit operatively coupled to the EEG acquisition device. The data processing unit may be configured to receive digitized EEG signals time-locked to the presented auditory stimuli via trigger information, extract from the digitized EEG signals a series of time-locked epochs, each epoch corresponding to at least one auditory stimulus, subdivide each epoch into a plurality of segments for subsequent feature analysis using a segmentation process to produce segmented data, and store the segmented data in a structured format for subsequent feature extraction and analysis.

### CROSS-REFERENCE TO RELATED APPLICATIONS

This application claims the benefit of priority of U.S. Provisional Patent Application Ser. No. 63/766,051, filed on Mar. 3, 2025, the entire content of which is hereby incorporated by reference herein.

### FIELD

The present disclosure relates to signal processing techniques for analyzing brain activity, particularly in the context of auditory evoked potentials and cognitive assessment.

### BACKGROUND

Electroencephalography (EEG) allows for the measurement of brain responses to external stimuli, known as evoked potentials. Auditory-evoked potentials (AEPs) are time-locked EEG responses to auditory stimuli, reflecting the brain's processing of sound. These responses provide insight into the functional integrity of the auditory system and may serve as indicators of a patient's cognitive status. Unlike neuropsychological tests, which can be influenced by language proficiency or educational background, AEP measurements are non-invasive, cost-effective, and free from cultural or educational biases. This makes them particularly valuable for standardized assessments of conditions such as Alzheimer's disease (AD).

Cognitive impairment, ranging from mild cognitive impairment (MCI) to dementia, is known to alter the morphology of AEP waveforms. Historically, research has focused on analyzing individual AEP components to evaluate their potential as diagnostic biomarkers for cognitive impairment, primarily by examining variations in amplitude and latency.

Despite their promise, clinical assessment of AEPs is hindered by limited specificity and the absence of standardized analytical methodologies. Variability in AEP responses across individuals-driven by factors such as age, hearing ability, and comorbid conditions-further complicates their utility as reliable biomarkers.

### SUMMARY

Embodiments of the present invention address and overcome one or more of the above shortcomings and drawbacks.

In an exemplary embodiment, a system for segmenting auditory-evoked potentials (AEPs) from electroencephalography (EEG) signals comprises an auditory stimulator configured to present controlled auditory stimuli to a subject, an EEG acquisition device including a plurality of sensors adapted to detect electrical brain signals from a scalp of the subject in response to the auditory stimuli, and a data processing unit operatively coupled to the EEG acquisition device.

In an exemplary embodiment the data processing unit is configured to receive digitized EEG signals time-locked to the presented auditory stimuli via trigger information, extract from the digitized EEG signals a series of time-locked epochs, each epoch corresponding to at least one auditory stimulus, subdivide each epoch into a plurality of segments for subsequent feature analysis using a segmentation process to produce segmented data, and store the segmented data in a structured format for subsequent feature extraction and analysis.

In an exemplary embodiment, the segmentation process comprises selecting user-defined segmentation parameters. In an exemplary embodiment, the segmentation parameters include at least one segmentation mode. In an exemplary embodiment, the segmentation process comprises generating segments from each epoch according to the at least one segmentation mode.

In an exemplary embodiment, the segmentation mode is selected from a group comprising sequential segmentation, shifting segmentation, expanding segmentation, or a combination thereof.

In an exemplary embodiment, the segmentation parameters further include a bin size, a shifting step size, an expanding step size, and/or a segmentation center point. In sequential segmentation, the epoch is divided into non-overlapping segments of fixed or variable lengths that collectively cover a post-stimulus time window. In shifting segmentation, a sliding window of user-defined fixed length moves across the epoch in overlapping increments defined by the shifting step size. In expanding segmentation, segments are generated that cumulatively extend outward from a predetermined central time point by increments defined by the expanding step size.

In an exemplary embodiment, the auditory stimulator comprises a digital signal processor (DSP) configured to transmit a synchronization signal or trigger code to the EEG acquisition device, thereby marking an onset time of each auditory stimulus.

In an exemplary embodiment, the EEG acquisition device includes an analog-to-digital converter and low-noise amplifiers configured to filter and digitize the electrical brain signals at a sampling rate of at least 500 Hz.

In an exemplary embodiment, the data processing unit is further configured to apply one or more filters to the digitized EEG signals prior to epoch extraction. In an exemplary embodiment, the filters comprising at least one of a band-pass filter to isolate a frequency range containing cognitive event-related responses or a notch filter to remove line-noise interference.

In an exemplary embodiment, the data processing unit is configured to remove artifacts from each epoch based on at least one criterion selected from the group consisting of a peak-to-peak amplitude threshold, a flatline detection threshold, and an independent component analysis (ICA) approach to remove ocular or muscle artifacts.

In an exemplary embodiment, the data processing unit is further configured to iteratively adjust the segmentation parameters based on classification accuracy feedback from a machine-learning module, so as to automatically determine an optimal set of segmentation parameters for feature extraction.

In an exemplary embodiment, a method of analyzing EEG signals associated with auditory stimuli to identify features of auditory-evoked potentials (AEPs), comprises the steps of presenting one or more auditory stimuli to a subject while recording EEG signals, associating each auditory stimulus with a trigger code indicative of a stimulus onset, extracting from the recorded EEG signals a plurality of time-locked epochs, each epoch corresponding to at least one auditory stimulus, segmenting each epoch into a plurality of segments, and extracting features from each segment for subsequent analysis.

In an exemplary embodiment, the step of segmenting each epoch further comprises receiving user-defined segmentation parameters including at least one segmentation mode and generating segments according to the at least one segmentation mode.

In an exemplary embodiment, the segmentation mode is selected from a group comprising sequential segmentation, shifting segmentation, expanding segmentation, or a combination thereof.

In an exemplary embodiment, the segmentation parameters further comprise a bin size, a shifting step size, an expanding step size, and/or a segmentation center point.

In an exemplary embodiment, sequential segmentation divides the epoch into non-overlapping segments, shifting segmentation generates overlapping segments by moving a fixed-length window in increments, and expanding segmentation produces cumulative segments expanding from a central time point.

In an exemplary embodiment, the step of segmenting each epoch into a plurality of segments further comprises iteratively performing segmentation with varying parameter values and selecting segmentation parameters based on an evaluation of classification performance from a machine-learning model trained on features extracted from the segments.

In an exemplary embodiment, the method further comprises a step of extracting one or more features from each segment, wherein the features include at least one of average amplitude, minimum amplitude, maximum amplitude, or peak-to-peak amplitude range within the segment, slope or rate of change in voltage over time within the segment, latency measurements including the time of a maximum or minimum deflection relative to the stimulus onset, area under an amplitude-versus-time curve indicating total evoked response, frequency-domain metrics derived via Fourier or wavelet transforms, including band power (delta, theta, alpha, beta) or spectral entropy, and event-related potential component metrics if present in the segment.

In an exemplary embodiment, the recording of EEG signals in step (a) is preceded by a filtering step. In an exemplary embodiment, the filtering step comprises at least one of applying a band-pass filter to reduce slow drifts and high-frequency noise, applying a notch filter to remove line noise, and down-sampling the filtered signals to a lower sampling rate after recording to facilitate subsequent segmentation.

In an exemplary embodiment, the method further comprises training a machine-learning model using the features extracted from each segmented epoch to classify whether the subject exhibits a cognitive impairment.

In an exemplary embodiment, a non-transitory computer-readable medium storing instructions which, when executed by one or more processors, cause the processors to perform a method for segmenting and structuring auditory-evoked potentials (AEPs) from EEG data.

In an exemplary embodiment, the method comprises receiving raw EEG data recorded while a subject is presented with at least one auditory stimulus, identifying trigger codes in the raw EEG data to demarcate stimulus onset times, extracting, based on the trigger codes, a set of time-locked epochs, and subdividing each epoch into a plurality of segments according to configurable segmentation parameters.

In an exemplary embodiment, the segmentation parameters include at least one segmentation mode.

In an exemplary embodiment, the segmentation mode is selected from a group comprising sequential segmentation, shifting segmentation, expanding segmentation, or a combination thereof.

In an exemplary embodiment, the segmentation parameters further comprise a bin size, a shifting step size, an expanding step size, and a segmentation center point.

In an exemplary embodiment, the segments are generated as non-overlapping segments in a sequential mode, overlapping segments via a sliding window in a shifting mode, or cumulatively expanding segments in an expanding mode.

In an exemplary embodiment, the instructions further cause the processors to execute at least one artifact rejection routine that removes epochs containing artifacts exceeding a predefined threshold prior to segmentation.

In an exemplary embodiment, the instructions further cause the processors to apply at least one filtering operation to the raw EEG data prior to epoch extraction, the filtering operation comprising a band-pass filter, a high-pass filter, a low-pass filter, or a notch filter.

In an exemplary embodiment, the instructions further cause the processors to store data from the plurality of segments in a structured arrangement that associates each segment with at least one time index, amplitude value, or frequency-domain representation.

In an exemplary embodiment, the instructions optionally provide for computing one or more segment-specific metrics, including amplitude statistics, slope measurements, or spectral power estimates, and outputting said metrics for display or subsequent analysis.

### BRIEF DESCRIPTION OF THE DRAWINGS

The foregoing and other aspects of the present invention are best understood from the following detailed description when read in connection with the accompanying drawings. For the purpose of illustrating the invention, these are shown in the drawings embodiments that are presently preferred, it being understood, however, that the invention is not limited to the specific instrumentalities disclosed. Included in the drawings are the following Figures:

FIG. 1 depicts a schematic representation of a system for processing AEPs, according to an exemplary embodiment of the present disclosure.

FIG. 2 depicts a system architecture for auditory stimulus presentation and EEG signal acquisition, comprising an electronic module that delivers auditory stimuli, an EEG amplifier that records brain responses via sensors, and a data processing pipeline for filtering, segmentation, and cognitive analysis, according to an exemplary embodiment of the present disclosure.

FIG. 3 depicts the data processing pipeline for AEP analysis, including raw EEG data acquisition, filtering, epoching, artifact rejection, and variable-size segmentation, with different segmentation approaches applied to the EEG waveform, according to an exemplary embodiment of the present disclosure.

FIG. 4 depicts the time-locked relationship between an auditory stimulus and the corresponding AEP in the EEG signal, highlighting the trigger point for stimulus presentation and the resulting neural response, according to an exemplary embodiment of the present disclosure.

FIG. 5 depicts different segmentation strategies applied to AEP waveforms, including coarse, fine-grained, and high-resolution segmentations, allowing for detailed temporal analysis, according to an exemplary embodiment of the present disclosure.

FIG. 6 depicts shifting and expanding time window segmentation techniques, where shifting segmentation moves fixed-size window(s) across the AEP signal in small increments, and expanding segmentation progressively increases the window size to capture additional portions of the response, according to an exemplary embodiment of the present disclosure.

FIG. 7 depicts the process of extracting features from segmented AEP waveforms, where various statistical and spectral features are computed from individual segments and subsequently used as input for a machine learning classifier, according to an exemplary embodiment of the present disclosure.

FIG. 8 depicts the extraction of multiple numerical features from segmented AEP waveforms, where individual data points within each segment are labeled and analyzed for their amplitude values, allowing for further computation of statistical and spectral characteristics for machine learning classification, according to an exemplary embodiment of the present disclosure.

FIG. 9 depicts an exemplary system architecture for cognitive impairment detection, illustrating the process from auditory stimulation and EEG signal acquisition to variable-size segmentation and machine learning classification, which determines whether the subject exhibits normal cognition or cognitive impairment, according to an exemplary embodiment of the present disclosure.

FIG. 10 depicts a flow diagram outlining the process for AEP data acquisition, and machine learning-based classification for cognitive assessment, according to an exemplary embodiment of the present disclosure.

FIG. 11 depicts different EEG sensor configurations, including full-head sensor coverage, temporal-central sensor positioning, and a headband covering frontal, temporal, and occipital areas, according to exemplary embodiments of the present disclosure.

FIG. 12 depicts a master flow diagram outlining an exemplary step-by-step process for EEG data acquisition, preprocessing (filtering, epoching, artifact rejection), variable-size segmentation, feature extraction, and machine learning classification to determine cognitive status as either normal or impaired, according to an exemplary embodiment of the present disclosure.

FIG. 13 depicts Five different auditory stimulation paradigms were used (steps) including different deviant stimuli differing in duration, frequency or varying interstimulus interval.

FIG. 14 depicts raw EEG traces with vertical lines representing the triggers marking the onset and offset of auditory stimuli.

FIG. 15 depicts an AEP segment with a size of 200 ms and extracted features marked in the deviant stimulus.

FIG. 16 depicts selected grand averaged AEP with a 95% confidence interval dataset of 3 Alzheimer's Disease (AD, gray) patients and 4 healthy volunteers (HV, black) recorded from temporal EEG electrodes T7 and T8. Results show different AEP amplitudes for specific AEP segments (see arrows) in the multi-feature auditory memory, paired-chirp and speech paradigm.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm. Further, referring to ranges between certain values, from a first value to a second value, means the endpoints are to be included.

The singular forms “a,” “an,” and “the” include plural referents unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “patient” and “subject” are interchangeable and may be taken to mean any living organism which may be treated with compounds of the present invention. As such, the terms “patient” and “subject” may include, but are not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child, or infant. In some embodiments, the patient or subject is a human.

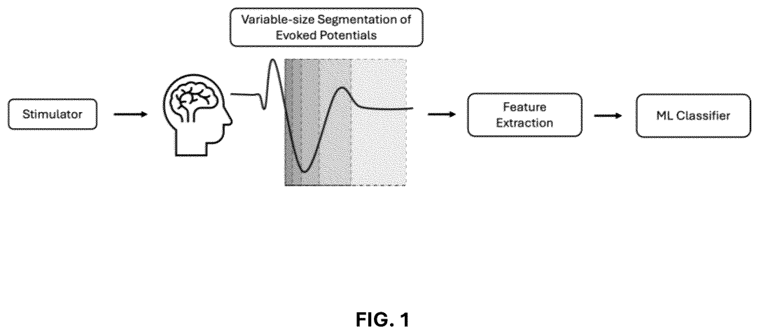

The systems and methods described herein identify features in evoked brain responses by employing a segmentation technique that allows for an in-depth analysis of auditory-evoked potentials (AEPs). FIG. 1 depicts a schematic representation of a system for processing AEPs.

In some embodiments, an EEG device is used alongside an electronic module with a stimulator that delivers auditory stimuli to a test subject. The electronic module may be synchronized with the portable EEG component through the delivery of trigger signals from a digital signal processor (DSP) to the EEG amplifier. Sensors connected to the EEG component are placed on the test subject's head, and the measured signals undergo amplification, filtering, and analog-to-digital conversion at a high sampling rate. The digitized EEG signal is then transmitted to an external device, such as a desktop computer, tablet, or smartphone, for further analysis.

In some embodiments, recorded responses undergo filtering, epoching, and segmentation into various bins of different sizes. Multiple features can be extracted from each AEP segment and subsequently used to train machine learning models for identifying cognitive abnormalities and assessing cognitive status.

Unlike conventional approaches that focus solely on amplitude and latency characteristics of individual AEPs, the systems and methods described herein extract multiple features, overcoming the limitations that hinder the clinical use of AEPs. Variable-size segmentation of AEPs recorded from the same subject can generate many distinct features, which can be used to train machine learning classifiers for detecting cognitive impairment and assessing cognitive function. In some embodiments, one or more AEPs can be recorded from the same test subject, different segmentation techniques with varying bin sizes can be applied.

Additionally, the described systems and methods may include a portable hardware component (including an electronic module and EEG amplifier) and a software architecture embedded in a computer, tablet, or digital processing module.

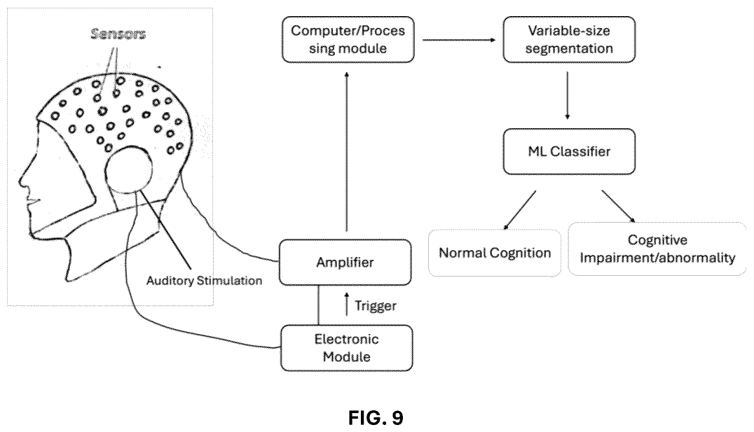

In an exemplary embodiment, the process begins when the user presses the start button on the electronic module, initiating auditory stimulation. With each auditory stimulus, a trigger code is sent to the amplifier to mark the EEG trace, facilitating the extraction of AEP epochs through the software component. The recorded EEG data is stored on an external computer, tablet, or digital signal processing module. The user can then utilize analysis software to process the EEG data into AEP epochs, perform segmentation, extract features, and train machine learning models. FIG. 9 provides an overview of such an exemplary embodiment.

### System Architecture (Hardware Components)

In some embodiments, the present invention relates to a system designed for AEP-based cognitive impairment detection.

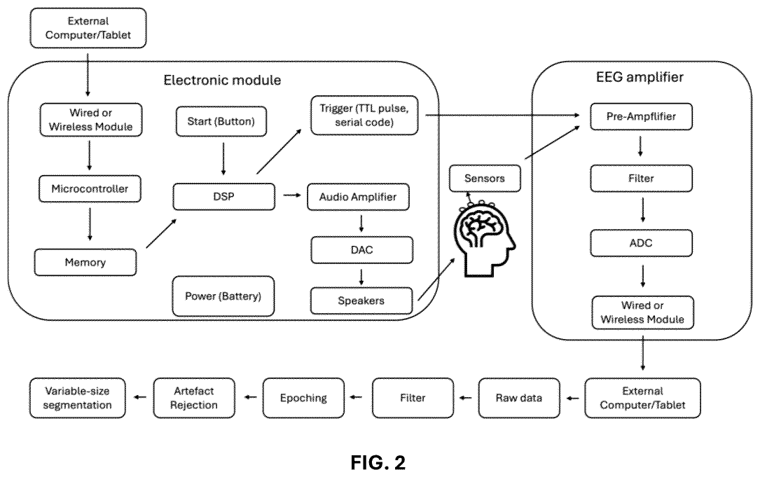

FIG. 2 illustrates an exemplary system for stimulus presentation and the collection of evoked responses using an electronic module for auditory stimulus generation and presentation, a portable EEG component, and a digital signal processing module embedded in or connected to the EEG component. In some embodiments, segmentation of the EEG signal is performed on an external computer, tablet, or digital signal processor.

The system and methodology allow for the collection and processing of auditory-evoked brain responses into smaller segments (in some embodiment, with variable sizes) for training machine learning classifiers. In some embodiments, the electronic module includes a digital-to-analog converter for presenting auditory stimuli. These stimuli may be loaded via a wireless or wired connection from an external computer or tablet to the electronic module. A microcontroller may write the auditory files and protocol table to the memory unit of the electronic module.

In some embodiments, a digital signal processor (DSP) can access the auditory stimuli and the protocol table and transmit these to the audio amplifier. The auditory stimuli may then be converted into an analog signal and delivered through a speaker to the subject's ears. Simultaneously, the DSP can send trigger codes, also stored in the protocol table, to the EEG amplifier. The EEG amplifier records the evoked activity from the sensors. The EEG signal may be pre-amplified, filtered, and digitized by an analog-to-digital converter (ADC) before being transmitted to a computer, tablet, or processing module.

In some embodiments, the computer, tablet, or processing module processes the raw EEG signal for further analysis. The processing steps include filtering, epoching, artifact rejection, and segmentation, generating distinct segments for each AEP. The segmentation process allows for the extraction of various features, which can serve as input data for training machine learning classifiers.

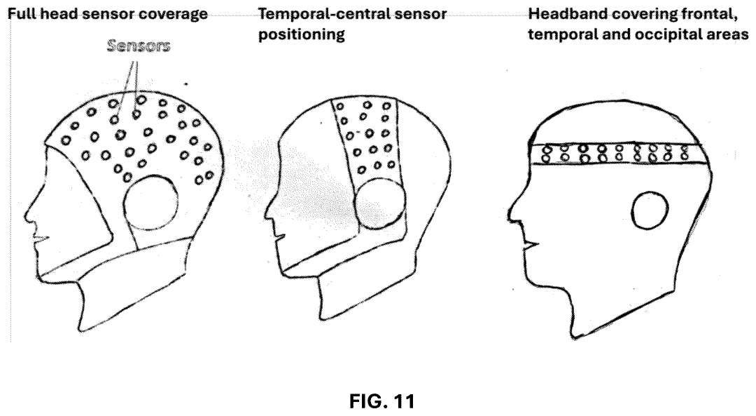

In some embodiments, the EEG amplifier is connected to sensors placed on the test subject's scalp. EEG sensors are a set of non-invasive electrodes placed on the scalp to record brain activity. FIG. 11 illustrates exemplary EEG sensor configurations. These sensors may include, but are not limited to, dry EEG electrodes or electrodes using ionic hydrogel to enhance signal quality. The EEG sensors may be incorporated into a cap covering the entire scalp or embedded into a headband that covers the frontal, temporal, and occipital brain regions.

In some embodiments, at least one active electrode is positioned at a location sensitive to auditory responses (e.g., the forehead at Fpz) along with a reference electrode (e.g., attached to the earlobe or mastoid) and a ground electrode. A multi-channel montage (e.g., including central sites such as Cz or frontocentral sites) may be used for enhanced spatial resolution. In some embodiments, even a single-channel setup near the forehead can be effective for cognitive impairment detection. The sensors capture tiny voltage fluctuations (~μV range) generated by the brain in response to auditory stimuli.

In some embodiments, EEG sensors may also be integrated into a structure covering the temporal, central, and parietal brain regions of the scalp. As a non-limiting example, the electrodes may include Ag/Cl-plated dry sensors with spiked, flexible extensions designed to facilitate signal acquisition in areas with dense hair.

The EEG sensors detect electrical activity on the test subject's scalp and transmit the signal to the EEG amplifier, where it undergoes filtering before being converted into a digital signal by an ADC. The recorded and digitized EEG signal can be stored locally (e.g., on an SD card or hard drive) or transmitted directly to a computer or processing module for further analysis.

In some embodiments, the EEG electrodes are connected to an electronic module that amplifies and digitizes the biosignals. In some embodiments, this module contains low-noise amplifiers designed to boost microvolt-level EEG signals while maintaining high gain and high input impedance to preserve signal fidelity. It also provides analog filtering to isolate the relevant frequency range of AEPs. As an example, for cortical AEPs, a band-pass filter of approximately 0.1-30 Hz may be applied, as most cognitive event-related potential (ERP) energy falls within this range. Low frequencies may be filtered to remove DC drift, while high frequencies may be filtered to reduce muscle artifacts and unrelated noise. After amplification and filtering, the ADC samples the signal at a sufficient rate (e.g., 500 Hz or 1000 Hz) and resolution (typically 16-bit or higher) to accurately capture the evoked potential waveform. The module may be a dedicated EEG acquisition unit or a wearable device, ensuring clean, digitized EEG data is available for processing.

In some embodiments, the system includes an auditory stimulus source to elicit AEPs. This may include speakers, headphones, insert earphones driven by an audio output (such as a sound card or tone generator) controlled by the system's processing unit. The auditory stimuli may be simple tone bursts, clicks/chirps, or more complex sounds, such as speech syllables or vowels, depending on the test paradigm. A trigger mechanism may be integrated to ensure synchronization between auditory stimulus presentation and EEG recording. For example, each time a sound is played, a synchronization signal may be generated. The auditory output hardware ensures consistent sound intensity and timing.

In some embodiments, a computing unit—such as a PC, laptop, tablet, or an embedded processor in a portable device—runs the software responsible for stimulus presentation, data acquisition, and signal processing. In some embodiments, this unit interfaces with the electronic module to receive digitized EEG data in real time. It also controls the timing of auditory stimuli and records event markers.

In some embodiments, the processing unit is a general-purpose computer connected via USB or a wireless connection to an EEG amplifier. In other cases, the processing unit may be a microcontroller or DSP integrated into a headband device, allowing for onboard processing. In some embodiments, the processing unit executes analytical algorithms, including filtering, segmentation, feature extraction, and classification.

In some embodiments, to ensure timing synchronization between stimulus presentation and EEG data acquisition, the system may include a trigger mechanism that marks each stimulus onset with a digital trigger signal recorded alongside the EEG. For example, in a PC-based setup, the software may send a pulse or event code to the EEG amplifier at the exact moment an auditory stimulus is presented. These triggers serve as time-stamped markers in the EEG recording, indicating the precise moment of stimulus onset (t=0) for each trial.

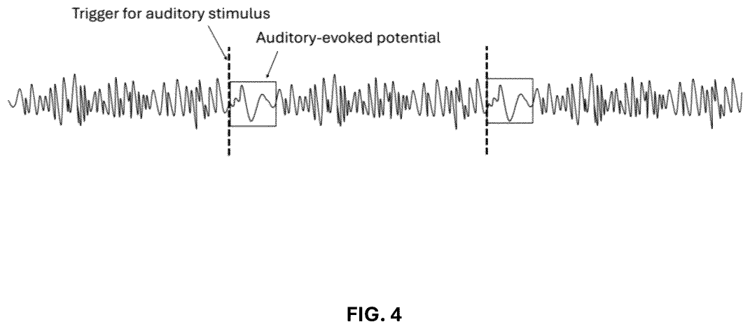

FIG. 4 illustrates this synchronization, represented by a sync line connecting the stimulus generator and the EEG module. This mechanism ensures that subsequent processing can accurately align EEG responses to their corresponding auditory stimuli. In some embodiments, the system achieves high temporal precision—on the order of milliseconds or better—allowing for the accurate segmentation of even fast brain responses.

Together, these hardware components form an integrated system in which the participant wears EEG sensors, hears a sequence of auditory stimuli, and the electronic module transmits amplified EEG signals to the processing unit. The processing unit manages stimulus delivery and captures synchronized EEG data for subsequent analysis. FIGS. 2 and 9 provide a visual representation of these system components and their interconnections in an exemplary embodiment.

### Stimulus Presentation and EEG Data Acquisition

In some embodiments, an AEP-based cognitive assessment involves presenting controlled auditory stimuli while simultaneously recording the subject's EEG.

The system comprises an electronic module with an integrated auditory stimulator, a memory unit, a processor, EEG sensors, and an EEG amplifier synchronized to the electronic module.

In some embodiments, the electronic module delivers auditory stimuli to the test subject's ear and transmits trigger codes to the EEG amplifier. An external computer or tablet may send the audio files and a corresponding table containing details such as audio file names, trigger codes, and interstimulus intervals via a wired or wireless connection (e.g., Bluetooth, Wi-Fi) to the electronic module.

In some embodiments, a protocol table defines the auditory stimulus sequence presented to the test subject. Each auditory stimulus may be associated with a unique trigger code to facilitate the extraction and analysis of AEP epochs. The interstimulus interval (ISI) specifies the time between two auditory stimuli. For example, the protocol may dictate that stimulus 1 is presented, followed by a 1000 ms ISI before stimulus 2 is delivered.

In some embodiments, the memory unit stores the auditory files (e.g., in .wav format) and protocol tables containing the stimulus sequence, trigger codes, and interstimulus intervals.

In some embodiments, a processor connected to the memory unit can retrieve the auditory stimulus files and protocol information.

In some embodiments, the DSP transmits the auditory files to the stimulator for stimulus delivery while simultaneously sending a trigger code to the EEG amplifier to mark the exact onset of auditory stimulus presentation (time 0).

In some embodiments, a microcontroller embedded in the electronic module transmits the auditory files and protocol table to the memory unit. Pressing the start button on the electronic module activates the DSP, which retrieves the auditory files and protocol sequence, sending them to the audio amplifier and stimulator according to the predefined protocol. The auditory stimuli may be delivered via transducers such as a speaker, insert earphones, or overhead headphones. At the onset of stimulus delivery, the DSP sends the associated trigger code to the EEG amplifier's preamplifier with millisecond accuracy.

In some embodiments, the recorded and digitized EEG signals contain trigger codes corresponding to the auditory stimuli presented to the test subject. A computer or processing module (e.g., a DSP) may include a software architecture for filtering the raw EEG signals using low-pass, high-pass, notch, or band-pass filters with specified frequency ranges.

In some embodiments, the DSP, embedded within the electronic module, can load the audio files and protocol table containing the audio file name, trigger code, and interstimulus interval from the memory unit. Upon activation, the DSP can transmit the audio files to the auditory stimulator for delivery of auditory stimuli. Simultaneously, a trigger code may be sent to the EEG amplifier to precisely mark the onset of auditory stimulus delivery.

In some embodiments, the trigger code may be transmitted as a separate EEG channel into the EEG amplifier or as a serial code. The separate EEG channel can serve as a reference marker that can be overlaid or merged with EEG traces during offline analysis to determine the exact timing of each auditory-evoked brain response. Each auditory stimulus type, and therefore each AEP type, may be assigned a unique trigger code. For example, an auditory stimulus of a 1000 Hz tone lasting 100 ms may be associated with trigger code “S115,” while a 1000 Hz tone lasting 50 ms may correspond to trigger code “S113.”

In some embodiments, EEG sensors placed on the scalp capture the brain's electrical activity during auditory stimulation. The signal may be transmitted to a pre-amplifier, where the signal-to-noise ratio can be enhanced before filtering is applied. The ADC then digitizes the filtered signal, which is transmitted via a wired or wireless module to an external computer, tablet, or digital processing module for analysis.

In some embodiments, recorded EEG signals undergo multiple preprocessing steps before variable-size segmentation. These steps may include filtering the EEG signal with high-pass, low-pass, notch, or band-pass filters to extract subcortical auditory-evoked responses (e.g., Frequency Following Responses, FFR), middle latency responses (e.g., P50 components), and long-latency responses (e.g., P100, Mismatch Negativity, or P300 components). In some embodiments, variable-size segmentation may also be applied to the frequency domain of the raw EEG data, generating multiple filtered EEG datasets using predefined frequency bins.

In some embodiments, the system can employ various auditory paradigms to evoke cognitive responses. A commonly used method is the auditory oddball paradigm, in which a sequence of repetitive “standard” tones is interspersed with infrequent “target” tones. The subject may be instructed to mentally count or attend to the target tones, eliciting the P300 wave—an event-related potential (ERP) component associated with attention and cognitive processing. Alternatively, the subject may be instructed to only passively listen to auditory stimuli during which the participant may or may not engage in another activity (for example, watch a silent movie with subtitles).

In some embodiments, each auditory stimulus may be presented multiple times during an auditory EEG recording session, allowing for the extraction of multiple AEP epochs per stimulus type.

Another paradigm used to elicit cognitive AEPs is mismatch negativity (MMN), which involves a sequence of auditory stimuli where infrequent “deviant” tones (differing in frequency, duration, or intensity) are interspersed within a series of repetitive “standard” tones. MMN can be elicited without active attention from the subject, making it particularly useful for assessing automatic auditory processing and cognitive function. More complex auditory stimuli, such as spoken words or syllables, may also be used to engage higher-order processing. The choice of paradigm and stimuli can be tailored to target specific cognitive functions.

In some embodiments, auditory stimuli are presented with a defined ISI to prevent overlapping brain responses and allow for clear identification of AEP components. As a non-limiting example, ISIs may range from 1 to 2 seconds. For instance, the system may present a tone followed by a 1000 ms interval before the next tone is delivered. This approach prevents overlap of consecutive AEPs and ensures that late cognitive components, such as P300 (which peaks around 300 ms and dissipates by ~600-700 ms), are fully captured before the next stimulus is introduced.

In some embodiments, the processing unit can generate auditory stimuli in structured trial blocks, such as 100-200 trials containing mostly standard tones interspersed with a smaller proportion of target or deviant tones, as seen in oddball and MMN paradigms. The stimulus type (standard or deviant) and timing for each trial can be controlled and randomized or fixed according to the experimental design.

In some embodiments, as each auditory stimulus is presented, the system records a synchronized trigger in the EEG data stream, marking the event in real time. These triggers serve as time-locked references, designating stimulus onset (t=0) for each trial. The system ensures millisecond-level precision through either hardware-based TTL triggers or software-generated event markers aligned with the auditory output.

Throughout the stimulus presentation, EEG data may be continuously recorded by the electronic module and streamed to the processing unit. Recording typically begins before the first stimulus and continues until the last trial is completed, ensuring that all relevant brain activity is captured. In portable implementations, data may be stored locally for later upload or streamed wirelessly in real time to an external computer.

In some embodiments, the raw EEG data includes both spontaneous brain activity and the time-locked responses to auditory stimuli. The subject remains passive throughout the session, aside from any task-related instructions (e.g., “count the high-pitched tones” in an active oddball paradigm). The recording environment is controlled to minimize movement artifacts and external noise, ensuring high-quality EEG signals for subsequent analysis.

In some embodiments, once the stimulus sequence is completed (e.g., after N trials or a predetermined duration), the system stops EEG recording. At this stage, each auditory stimulus presentation is associated with a corresponding EEG epoch marked by trigger signals. These raw epochs may still contain ongoing EEG activity and noise; therefore, further signal processing is required to extract and analyze the AEPs. The acquired dataset consists of continuous EEG recordings with embedded event markers, ensuring alignment between auditory stimuli and the corresponding brain responses.

The data acquisition phase ensures the collection of time-locked EEG responses to known auditory events. The stimulus presentation methodology reliably elicits cognitive AEPs, such as MMN and P300, which serve as potential indicators of cognitive impairment. The precise synchronization of stimulus presentation and EEG recording allows for the isolation of these responses from background neural activity.

By the end of this phase, the system has captured a well-structured dataset that is ready for processing. The next steps involve segmenting the EEG signal into relevant epochs, applying signal enhancement techniques, and extracting meaningful features that can provide insight into cognitive function.

### Data Processing Pipeline (Preprocessing and Epoching)

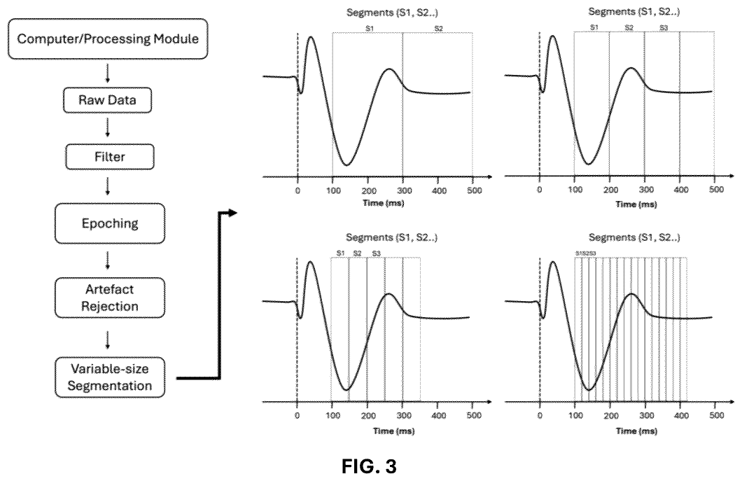

In some embodiments, once the raw EEG data containing embedded stimulus triggers is collected, the system executes a sequence of signal processing steps to prepare the data for analysis. FIG. 3 illustrates the processing of AEPs, including various segmentation approaches used to analyze distinct components of the evoked responses.

In some embodiments, an external computing device, such as a computer, tablet, or digital processing module, may be used to facilitate processing steps. In some embodiments, this device is equipped with a software architecture that allows for the automated processing of raw EEG data into structured epochs or segments. In some embodiments, once the data is transmitted to the external device, no further user interaction with the electronic module or EEG amplifier is required; the software can perform all necessary processing steps, ensuring efficiency and consistency in data analysis.

In some embodiments, the initial preprocessing of EEG data involves filtering to remove noise and irrelevant frequency components. The recorded EEG signals undergo multiple processing steps before segmentation is applied. These steps may include, but are not limited to, filtering with high-pass, low-pass, notch, or band-pass filters to isolate specific auditory-evoked responses. Different frequency bands may be targeted to extract subcortical responses (e.g., frequency-following responses, FFR), middle latency responses (e.g., P50 components), and long-latency responses (e.g., P100, mismatch negativity, or P300 components). Additionally, segmentation may be applied in the frequency domain by creating multiple filtered EEG datasets with pre-defined frequency bins.

A band-pass filter may be applied to isolate the typical ERP frequency range. In some embodiments, the data is band-pass filtered between approximately 0.5 Hz and 30 Hz, as cognitive ERP components (e.g., N100, P200, N200, P300) predominantly fall within this range. This filtering process attenuates slow drifts (e.g., baseline wander) and removes high-frequency noise from muscle activity or power-line interference, improving the signal-to-noise ratio. Additionally, a notch filter at 50/60 Hz may be applied to eliminate mains interference, if present. Filtering can be performed in real-time during data acquisition or applied offline after recording.

After filtering, the EEG signal primarily contains neural activity relevant to the auditory response. In some embodiments, the filtered EEG data is down sampled to a lower rate (e.g., from 1000 Hz to 250 Hz) to optimize processing efficiency while preserving critical ERP waveform features.

As illustrated in FIG. 4, the recorded trigger timestamps allow segmentation of the continuous EEG data into epochs corresponding to individual stimulus events. In some embodiments, the filtered signal is segmented into epochs containing the AEP response. Each trigger code corresponds to a specific auditory stimulus, allowing for the extraction of epochs based on these markers. The extracted epochs can be stored separately for each AEP type.

Each epoch represents a time-locked slice of the EEG surrounding a stimulus event. For example, the system may extract epochs spanning 200 ms before stimulus onset (to establish a baseline) to 600 ms after onset. In some embodiments, these epochs can be aligned to the precise moment of stimulus presentation. Alternatively, these epochs can be aligned to specific timepoints before or after stimulus presentation. In some embodiments, EEG data outside the epoch windows is excluded or stored separately for further analysis. At this stage, the dataset is structured as a series of time-locked trials rather than a continuous EEG recording.

In some embodiments, to ensure consistency across epochs, a baseline correction can be applied. Typically, the mean EEG voltage during a pre-stimulus period (e.g., −100 to 0 ms) is computed for each epoch. This mean value can then be subtracted from the entire epoch trace, zero-centering the data at stimulus onset and eliminating any pre-existing voltage offsets or slow drifts. Following baseline correction, any post-stimulus deviations can be attributed to the auditory stimulus rather than extraneous voltage variations.

Even with careful recording, some EEG epochs may be contaminated by artifacts, such as blinks, muscle activity, or momentary electrode disconnections. In some embodiments, the system automatically detects and excludes or corrects these artifacts. One approach involves peak-to-peak amplitude thresholding, where an epoch is marked as invalid if its voltage exceeds a predefined range (e.g., ±50 μV), as typical AEPs are much smaller (a few μV), and large deviations generally indicate noise or motion artifacts. Additionally, the system may monitor channels for flatlining or signal saturation. Other artifact rejection techniques include electrooculogram (EOG) monitoring for eye movement detection or independent component analysis (ICA) to remove ocular and muscle-related noise.

After artifact rejection, the remaining “clean” epochs contain valid AEP responses. If too many epochs are rejected, the system may prompt the user to collect additional data or indicate that the results may be less reliable due to insufficient valid trials.

In some embodiments, the system computes an average AEP waveform by averaging all clean epochs for a given condition. Averaging improves the signal-to-noise ratio by reducing random EEG fluctuations and revealing the consistent evoked response. For example, averaging over 50 trials typically can result in a clearer P300 peak than a single-trial waveform.

In some embodiments, the system may use the averaged AEP for feature extraction, while in others, it may retain single-trial information to analyze variability across trials. For example, if an auditory stimulus is presented 100 times, 100 AEP epochs are recorded. After artifact rejection, approximately 90% of trials may remain valid (e.g., 90/100 AEP epochs). The cleaned epochs can either be averaged into a single AEP waveform or analyzed individually for single-trial variability. Regardless of whether averaging is applied, the next step—segmentation—divides the waveform into meaningful temporal regions for further analysis.

Additionally, new AEP epochs may be derived by subtracting averaged responses to deviant auditory stimuli from those of standard auditory stimuli, such as in the computation of MMN. Once extracted, these epochs undergo segmentation based on predefined parameters, including segmentation type, bin size, and step size for expanding or shifting windows.

In some embodiments, instead of analyzing each epoch as a fixed time window, the epochs are subdivided into multiple segments of varying sizes for a more detailed analysis. The segmentation approach divides the AEP into smaller temporal sections, such as an early segment covering 0-100 ms, a mid-segment covering 100-300 ms, and a late segment covering 300-600 ms. Additionally, sliding windows and cumulative windows may be used to capture responses at different time scales.

By generating these sub-epochs, the system creates a rich dataset of time-aligned segments, each potentially capturing distinct ERP components or temporal patterns. These segments can be analyzed for various features, including amplitude, frequency, and latency, enabling a machine learning model to assess different time regions of the response rather than relying on a single global feature.

Unlike conventional ERP analysis, which typically examines a single post-stimulus window or a fixed latency point, variable-size segmentation allows for overlapping and non-uniform segment lengths. This flexibility enhances the ability to detect cognitive impairment by ensuring that abnormalities occurring at different time scales or in different portions of the response are not overlooked.

After these processing steps, the raw EEG data can be transformed into a structured dataset of clean, segmented AEPs. Each segment may be time-locked to the stimulus and correspond to a specific portion of the evoked response. The segmented data is now ready for feature extraction.

### Segmentation Techniques

Segmentation involves dividing each AEP epoch into sub-epochs of varying lengths and positions in time. This approach accounts for the possibility that cognitive impairment may influence different portions of the AEP waveform to different degrees. For example, late cognitive components may be affected more significantly than early sensory components. By exploring multiple segment sizes and positions, the system does not rely on a single predefined epoch window but instead allows the data to determine where discriminative features emerge.

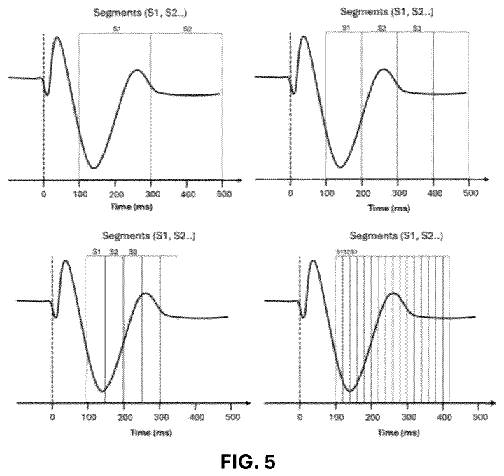

In some embodiments, the cleaned AEP epochs (or the average AEP epoch) are segmented using bins of varying sizes. As a non-limiting example, bins can range from 1, 5, 10, 20, 50, 100, or 250 ms. FIG. 5 illustrates segmenting into bins of different sizes.

In sequential segmentation, the AEP epoch is divided into non-overlapping segments of different sizes. In FIG. 5, for example, the top left panel depicts a basic two-segment division (S1, S2), where the entire response is split into broad intervals. The top right panel increases the segmentation resolution by dividing the epoch into three intervals (S1, S2, S3), providing more granular temporal analysis. The bottom left panel further refines the segmentation, dividing the epoch into smaller, equally spaced time bins. The bottom right panel illustrates a high-resolution segmentation approach, where the AEP is divided into numerous fine-grained segments, allowing for detailed temporal pattern analysis.

The following example demonstrates the effect of grouping raw EEG data into bins. Table 1 presents a single raw EEG epoch with 10 values, sampled at 1000 Hz.

TABLE 1

| Time (ms) | 1 | 2 | 3 | 4 | 5 | 6 | 7 | 8 | 9 | 10 |
|---|---|---|---|---|---|---|---|---|---|---|
| Value | 0.34 | 0.56 | 0.98 | 1.5 | 1.2 | 1.1 | 0.97 | 1.2 | 1.34 | 1.56 |

Selecting a bin size of 5 ms leads to the extraction of 2 segments (Segment Nr 1 and Segment Nr 2), as depicted in Table 2.

TABLE 2

| | Segment Nr | | | | | | | | | |
|---|---|---|---|---|---|---|---|---|---|---|
| | 1 | 1 | 1 | 1 | 1 | 2 | 2 | 2 | 2 | 2 |
| Value | 1 | 2 | 3 | 4 | 5 | 1 | 2 | 3 | 4 | 5 |
| Index | | | | | | | | | | |
| Time | 1 | 2 | 3 | 4 | 5 | 6 | 7 | 8 | 9 | 10 |
| (ms) | | | | | | | | | | |
| Value | 0.34 | 0.56 | 0.98 | 1.5 | 1.2 | 1.1 | 0.97 | 1.2 | 1.34 | 1.56 |

Each segment contains 5 values which can be indexed from 1-5 for analysis purposes. Alternatively, as depicted in Table 3, using a bin size of 2 ms creates 5 segments with two values each.

TABLE 3

| | Segment Nr | | | | | | | | | |
|---|---|---|---|---|---|---|---|---|---|---|
| | 1 | 1 | 2 | 2 | 3 | 3 | 4 | 4 | 5 | 5 |
| Value | 1 | 2 | 1 | 2 | 1 | 2 | 1 | 2 | 1 | 2 |
| Index | | | | | | | | | | |
| Time | 1 | 2 | 3 | 4 | 5 | 6 | 7 | 8 | 9 | 10 |
| (ms) | | | | | | | | | | |
| Value | 0.34 | 0.56 | 0.98 | 1.5 | 1.2 | 1.1 | 0.97 | 1.2 | 1.34 | 1.56 |

These different sequential segmentation strategies ensure that the entire AEP is covered in distinct intervals, allowing for comparisons across different time windows. By analyzing how features evolve across different segments, the system can identify which portions of the AEP waveform best differentiate between normal and impaired cognitive function.

In addition to sequential segmentation, variable-size segmentation may use shifting or expanding time windows to capture changes in AEP patterns over time.

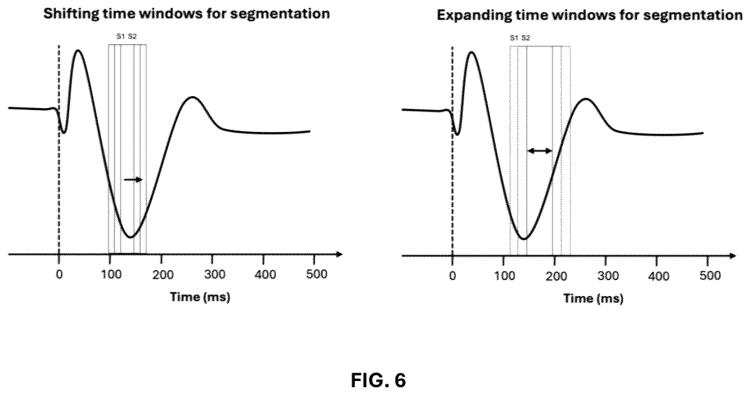

FIG. 6 presents two additional segmentation techniques: shifting and expanding time windows.

The left panel of FIG. 6 depicts shifting segmentation. In this approach, a fixed-size window (e.g., 2 ms) moves across the AEP epoch in small increments (e.g., 1 ms), creating overlapping segments. Each new segment shifts by a predefined step size, capturing slightly different portions of the waveform to ensure no key temporal information is overlooked. This overlapping strategy allows for a high-resolution analysis of how signal features evolve over time. Table 4 demonstrates this approach, where each segment contains two data points, and the segmentation process generates overlapping segments by shifting the window in increments of 1 ms. This method is particularly useful for identifying subtle variations in AEP features across different time regions, making it an effective tool for detecting cognitive impairment.

TABLE 4

| Segment Nr | Value Index | Time (ms) | Value |
|---|---|---|---|
| 1 | 1 | 1 | 0.34 |
| 1 | 2 | 2 | 0.56 |
| 2 | 1 | 2 | 0.56 |
| 2 | 2 | 3 | 0.98 |
| 3 | 1 | 3 | 0.98 |
| 3 | 2 | 4 | 1.5 |
| 4 | 1 | 4 | 1.5 |
| 4 | 2 | 5 | 1.2 |
| 5 | 1 | 5 | 1.2 |
| 5 | 2 | 6 | 1.1 |
| 6 | 1 | 6 | 1.1 |
| 6 | 2 | 7 | 0.97 |
| 7 | 1 | 7 | 0.97 |
| 7 | 2 | 8 | 1.2 |
| 8 | 1 | 8 | 1.2 |
| 8 | 2 | 9 | 1.34 |
| 9 | 1 | 9 | 1.34 |
| 9 | 2 | 10 | 1.56 |

The right panel of FIG. 6 depicts expanding segmentation, in which an initial small window expands incrementally in both forward and backward directions from a central time point. Each successive segment includes more of the post-stimulus response, allowing for cumulative analysis. Expanding segmentation is useful for identifying the duration over which a specific AEP component remains relevant for classification and detecting cognitive impairment. Table 5 illustrates this approach with an expanding window centered at 5 ms, growing outward in 1 ms increments until it encompasses the full epoch. This technique helps determine the optimal time range for feature extraction by analyzing whether a longer response window enhances classification accuracy. By examining different expansion strategies, this method ensures that both early and late response components are thoroughly analyzed.

TABLE 5

| Segment Nr | Value Index | Time (ms) | Value |
|---|---|---|---|
| 1 | 1 | 5 | 1.2 |
| 2 | 1 | 4 | 1.5 |
| 2 | 2 | 5 | 1.2 |
| 2 | 3 | 6 | 1.1 |
| 3 | 1 | 4 | 0.98 |
| 3 | 2 | 5 | 1.5 |
| 3 | 3 | 6 | 1.2 |
| 3 | 4 | 7 | 1.1 |
| 3 | 5 | 4 | 0.97 |
| . . . | . . . | . . . | . . . |

In some embodiments, an alternative approach may combine a shifting with an expanding window. For example, as depicted in Table 6, below, a window may be centered at 5 ms with a bin size of 2 ms (from 5-6 ms) and may shift by 1 ms and then expand by 1 ms in both directions.

TABLE 6

| Segment Nr | Value Index | Time (ms) | Value |
|---|---|---|---|
| 1 | 1 | 5 | 1.2 |
| 1 | 2 | 6 | 1.1 |
| 2 | 1 | 6 | 1.1 |
| 2 | 2 | 7 | 0.97 |
| 3 | 1 | 5 | 1.2 |
| 3 | 2 | 6 | 1.1 |
| 3 | 3 | 7 | 0.97 |
| 3 | 4 | 8 | 1.2 |
| . . . | . . . | . . . | . . . |

In some embodiments, the segmentation may involve first shifting the window in one direction and then expanding the window size to extract a second segment. For example, as depicted in Table 7, a bin size of 2 ms can be used, centered at 3 ms, expanded by 1 ms in both directions and then shifted by 1 ms in one direct to extract a second segment.

TABLE 7

| Segment Nr | Value Index | Time (ms) | Value |
|---|---|---|---|
| 1 | 1 | 3 | 0.98 |
| 1 | 2 | 4 | 1.5 |
| 2 | 1 | 2 | 0.56 |
| 2 | 2 | 3 | 0.98 |
| 2 | 3 | 4 | 1.5 |
| 2 | 4 | 5 | 1.2 |
| 3 | 1 | 3 | 0.98 |
| 3 | 2 | 4 | 1.5 |
| 3 | 3 | 5 | 1.2 |
| 3 | 4 | 6 | 1.1 |

These segmentation techniques-sequential, shifting, and expanding-can be applied independently or in combination to extract a comprehensive set of time-dependent features from the AEP waveform. By casting a wide net across different temporal resolutions, this approach ensures that meaningful AEP variations are captured, maximizing the sensitivity of cognitive impairment detection.

In sequential segmentation, the epoch is divided into consecutive, non-overlapping segments that systematically cover the entire response window. Each segment captures a distinct temporal portion of the AEP, allowing for a structured partitioning of the evoked response. The segment boundaries may be determined based on equal-duration intervals or aligned with known ERP component timings (e.g., one segment may center around the N100/P200 timeframe, another around N200/P300). For example, if a 10-sample EEG epoch is segmented using a bin size of 5 ms, the segmentation would produce two distinct non-overlapping segments, as illustrated in Table 2. Similarly, if the bin size is reduced to 2 ms, the number of segments increases, allowing for finer temporal resolution, as demonstrated in Table 3.

The significance of sequential segmentation is that it ensures full coverage of the AEP in distinct, non-overlapping intervals, allowing for the identification of impairment-related differences across time. By systematically partitioning the AEP, this approach allows for a detailed temporal analysis, ensuring that changes in neural activity occurring at different points in the response window can be independently assessed. For instance, analysis may reveal that abnormalities are more pronounced in the later segments compared to earlier ones. Additionally, this approach preserves the temporal ordering of features, enabling comparisons between early sensory responses (e.g., P50/N100/P200) and later cognitive responses (e.g., P300). Sequential segmentation effectively creates multiple regions of interest within the AEP, offering a more granular perspective than conventional analyses that focus on a single predefined window (e.g., only analyzing P300 amplitude). By segmenting the epoch in this manner, the classifier can assess the relative contributions of different time regions, ensuring that meaningful variations in AEP features are not overlooked.

In shifting segmentation, a sliding window moves across the epoch in overlapping steps, generating multiple, overlapping segments of a fixed length. Each segment shifts by a predefined step size, allowing for a more detailed temporal analysis of the AEP waveform. Unlike sequential segmentation, where each segment is independent and non-overlapping, shifting segmentation ensures that each time point in the AEP is included in multiple segments, increasing the likelihood of capturing transient events and subtle changes in the signal. For example, as demonstrated in Table 4, if a bin size of 2 ms is used with a shifting step of 1 ms, nine overlapping segments are created, each capturing slightly different portions of the EEG epoch. This technique is particularly effective when abnormalities occur at varying latencies, as it increases the probability of detecting variations across multiple time windows.

Shifting segmentation effectively generates a time series of local features, similar to a moving average or moving spectral analysis, which highlights transient variations in the AEP waveform. This method is especially useful when cognitive impairment affects the timing of peaks-such as a delayed P200 component-since a sliding window approach can track gradual peak shifts over time, whereas a fixed-segment approach might fail to capture such changes. Additionally, the overlapping nature of shifting segmentation introduces a degree of redundancy, which enhances the robustness of feature extraction by mitigating minor temporal misalignments. During the analysis phase, features from multiple overlapping windows may exhibit high correlation, requiring a feature selection step to retain only the most informative ones. By systematically capturing different portions of the waveform, shifting segmentation provides a comprehensive temporal analysis of the AEP, ensuring that no key differentiating feature is missed due to arbitrarily fixed epoch boundaries.

In addition to enhancing sensitivity to small waveform variations, shifting segmentation allows for a smoother representation of time-dependent neural activity. If a cognitive impairment leads to changes in AEP morphology, such as prolonged latency or reduced amplitude of specific components, shifting segmentation can capture these changes in a dynamic manner. For example, if a P200 peak occurs slightly later in an impaired group, shifting segmentation allows the classifier to detect the progressive shift in energy across the overlapping windows. This is particularly useful for analyzing ERPs where temporal precision is critical for classification.

Because shifting segmentation creates a large number of overlapping segments, the extracted features may contain redundant information. To mitigate this, a subsequent feature selection process may be used to filter out highly correlated features and retain only the most relevant ones. This step ensures that the classification model does not become biased by repetitive information while maintaining the advantages of high temporal resolution. By systematically reducing redundancy while preserving crucial AEP characteristics, the feature selection process optimizes model performance and interpretability.

Overall, shifting segmentation provides a high-resolution temporal analysis of the AEP waveform, ensuring that no potentially discriminative feature is overlooked due to arbitrary segmentation choices. By allowing each point in the EEG signal to be analyzed within multiple overlapping time windows, this method increases the likelihood of capturing abnormalities related to cognitive impairment.

In some embodiments, expanding segmentation takes a cumulative approach, creating segments that start at a central time point and progressively extend outward in both directions. Instead of defining fixed-length non-overlapping segments, this method generates successively longer windows that incrementally incorporate more of the post-stimulus period. For example, as demonstrated in Table 5, an expanding window centered at 5 ms extends incrementally by 1 ms in both directions until it reaches the full epoch length. This approach allows for a flexible, dynamic analysis of AEP responses, capturing both short- and long-duration patterns. FIG. 6 illustrates this method with nested segments, each growing in duration to progressively include additional portions of the AEP waveform.

The purpose of expanding segmentation is to analyze how the inclusion of successive response components influences signal characteristics and classification performance. Early segments (e.g., 0-100 ms) primarily capture brainstem and early cortical responses (e.g., wave I-V, P50, N100), whereas later (or longer latency) segments (e.g., 0-400 ms) incorporate additional cognitive components, such as the P300. By generating features across expanding windows, the system can determine at which time intervals group differences between normal and cognitively impaired subjects emerge most distinctly. This method provides insight into whether cognitive impairment affects only specific AEP components or whether changes occur across the entire response window.

For example, an analysis may find little distinction between impaired and normal groups when using only the first 300 ms of data, but clear differentiation when using 0-500 ms, which includes the P300 component. This finding would suggest that later cognitive processes, rather than early sensory responses, are more relevant for classification. Conversely, if an earlier expanding window already shows significant differences, it implies that early responses contribute meaningfully to the detection of cognitive impairment. Expanding segmentation can also help identify the optimal epoch length for classification-determining whether short or long-time windows provide the most informative features for distinguishing normal from impaired cognition.

Additionally, this method allows for an evaluation of the incremental contribution of additional temporal information. By systematically testing a series of expanding windows, the system can determine whether classification accuracy improves with longer segments or whether performance plateaus after a certain duration. If accuracy stabilizes beyond a certain time point, this suggests that including additional post-stimulus data does not enhance the ability to distinguish between groups. This insight can help simplify feature selection by focusing on the most informative temporal ranges, reducing computational complexity while maximizing classification performance.

Expanding segmentation can also be used to derive features that capture cumulative energy or the integrated response over different time intervals. For example, comparing the mean amplitude between the 0-300 ms and 0-600 ms windows can reveal differences in response dynamics. If cognitive impairment delays neural processing, the mean amplitude over the first 300 ms may be similar across groups, while the mean amplitude over 600 ms may differ, reflecting prolonged or weakened processing in impaired individuals. Additionally, integrating power across expanding windows allows the system to track changes in spectral characteristics over time, providing insights into how neural activity evolves within an AEP epoch.

This technique effectively analyzes the AEP in a progressively unfolding manner, allowing for a data-driven determination of optimal time windows for classification. By examining expanding windows, the system adapts to cases where the boundary between “early” and “late” responses is not predefined, ensuring that the selected analysis window is neither too short nor too long. Each expanding segment encapsulates “everything up to time T,” allowing the machine learning model to determine which time range is most informative for distinguishing cognitive conditions. This adaptive approach minimizes the risk of arbitrarily selecting a window that excludes relevant information, ensuring that both transient and sustained neural responses are captured for accurate classification.

These segmentation strategies—sequential (distinct consecutive windows), shifting (overlapping moving windows), and expanding (cumulative windows)—are not mutually exclusive. The system described herein can apply all three methods to the same dataset to generate a comprehensive set of segments. For instance, an initial broad sequential segmentation may be used to divide the AEP into distinct temporal regions, followed by a shifting segmentation approach to refine feature extraction within each region by introducing overlapping windows. Finally, an expanding window approach may be applied to validate optimal segment lengths, ensuring that both short- and long-duration patterns are captured.

This multi-faceted approach can result in dozens of segments per epoch, some overlapping and others nested, thereby capturing neural activity at multiple temporal resolutions. While this segmentation strategy generates a large number of candidate features, the subsequent feature selection process helps eliminate redundancy and retain only the most informative attributes. By analyzing AEPs across different time scales, this method minimizes the risk of overlooking key temporal patterns associated with cognitive impairment. In contrast, conventional ERP analysis often preselects only one or two fixed time windows (e.g., measuring only P300 amplitude or latency), potentially missing other valuable diagnostic features.

In sum, variable-size segmentation ensures comprehensive temporal coverage of the AEP, ranging from fine-grained local windows to broad cumulative windows. This adaptability allows for a more detailed characterization of both early and late cognitive components, improving the detection of subtle abnormalities in AEP responses. FIGS. 5-6 provide a visual summary of these techniques applied to an example AEP waveform, illustrating how each method partitions the signal in different ways. By employing a combination of segmentation strategies, the system maximizes the extraction of relevant neural features, allowing for a more accurate classification of cognitive function.

Feature Extraction from Segmented AEPs

After segmentation, each AEP epoch is divided into multiple segments (time slices), and the extracted segments of each bin size are stored for further analysis. Segmentation allows for the extraction of a wide range of features, which can be used to train machine learning models and optimize their performance.

The next step is to extract quantitative features from each segment, transforming the segmented EEG data into numerical descriptors that capture characteristics of the evoked response. The systems and methods described herein may employ various feature types to comprehensively describe each segment's signal shape, magnitude, and frequency content.

In some embodiments, the user can select different segmentation types and parameters, with the extracted features serving as training data for machine learning classifiers.

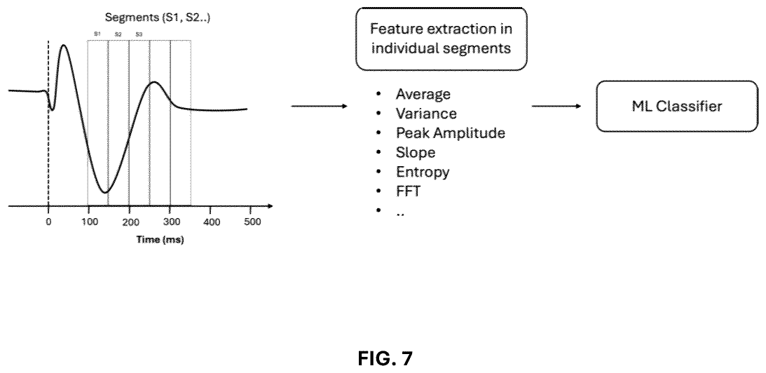

A variety of features can be extracted from each segment of each bin size, including, but not limited to, average amplitude, variance, peak amplitude, slope, entropy, and frequency-domain features such as Fourier transforms. FIG. 7 depicts the process of extracting features from segmented AEP waveforms. The left portion of the figure illustrates the segmentation of an AEP epoch into multiple non-overlapping time intervals (S1, S2, S3, etc.), allowing for a detailed temporal analysis of the evoked response. Each segment captures distinct characteristics of the AEP waveform, allowing for the extraction of meaningful statistical and frequency-based features. Once segmentation is complete, various quantitative features are extracted from each segment. After feature extraction, the numerical descriptors from each segment are compiled and used as inputs for machine learning (ML) classification. In some embodiments, a ML classifier processes these extracted features to differentiate between normal and impaired cognitive states based on learned patterns.

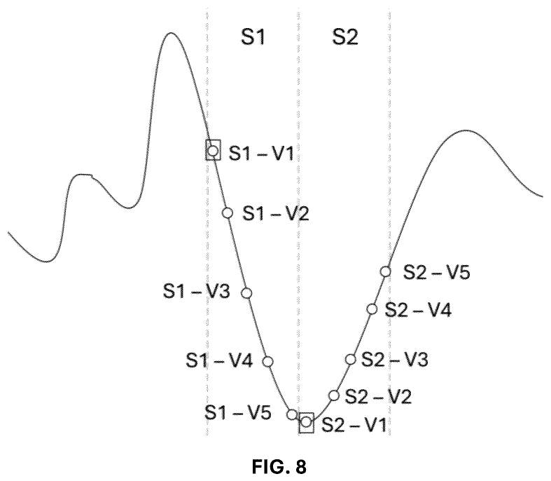

FIG. 8 illustrates the extraction of multiple features from various AEP segments. A combination of distinct features from different AEPs can be used as input for training machine learning classifiers. These features may be structured in matrices, rows, or columns. They may be derived from a single EEG channel or multiple EEG channels. Each segment may contain single or multiple values, which can be further processed to derive additional features. For example, the mean amplitude of an AEP epoch within a particular segment can be calculated by averaging the values within that segment. Similarly, a feature may be extracted that reflects the local minimum value in a specific segment. For instance, if the minimum value in segment 2 is S2-V1, the extracted feature may be labeled F_S2 MIN.

In some embodiments, certain features may be extracted across all AEP epochs. For example, the variance of the feature across different single AEP epochs of the same type may be computed, leading to the extraction of a new feature which reflects the variance of the averaged value in segment 1 across all AEP epochs.

For example, AEP, bin size, segment, and feature may all be extracted from a single EEG channel. In some embodiments, an additional dimension can be incorporated by selecting features from different EEG channels. A set of features may include the same feature recorded from distinct scalp locations or different features recorded from distinct scalp locations.

In some embodiments, time-domain amplitude features provide basic statistical measures of the EEG waveform within a segment. These features may include the mean amplitude (average voltage) over the segment, peak amplitude (maximum voltage), minimum amplitude (most negative voltage), and peak-to-peak range. These measures summarize the overall level and extreme values of the evoked response within the segment and are useful for capturing components that manifest as deflections in the waveform. For example, if a P300 wave occurs within a segment, the peak amplitude feature would capture its magnitude. By comparing mean or peak amplitudes across segments, the system can determine whether a specific portion of the response is stronger or weaker.

For instance, in an early segment (e.g., 0-100 ms), the mean amplitude might be near zero, indicating no significant response. In contrast, within a segment covering the P200 response, the mean amplitude could be positive due to the upward deflection characteristic of P200.

In some embodiments, latency and temporal features capture timing characteristics within the segment may be measured. If the segment is long enough to include a known ERP component (such as N100 or P300), the latency of that component's peak can be measured—i.e., the time (relative to the stimulus onset or segment start) at which the peak occurs. For example, if a segment spans 250-500 ms, it may contain the P300 peak, and the system can record that the maximum amplitude occurs at 380 ms within this window. In some embodiments, delayed peak latencies may be indicative of cognitive slowing.

Additionally, onset or offset latencies can be defined within segments, such as the time it takes for the voltage to deviate significantly from baseline after stimulus presentation or the duration for which activity within the segment persists. Another useful metric is the area under the curve (i.e., the integral of the EEG trace over the segment), which combines amplitude and duration to provide a measure of the total evoked response. This metric can differentiate between responses that are short and sharp versus those that are prolonged.

Furthermore, slope features can be derived, including the overall slope between the start and end of the segment (indicating the general trend) and the maximum instantaneous slope (steepest deflection) within the segment. These features reflect the dynamics of the response, such as how rapidly it rises or falls. If an ERP component is delayed or prolonged in an impaired brain, slope features can highlight a slower rise or a sustained response. Collectively, these time-based features help the model assess not only the magnitude of the response but also its timing and dynamics within each segment.

In some embodiments, the system analyzes the segment's signal in the frequency domain to capture information about its oscillatory content. Although AEPs are transient signals, they can be decomposed into frequency components using Fourier or wavelet transforms to determine how energy is distributed across different frequency bands. Features extracted from this analysis include band power in standard EEG frequency bands—such as delta (0.5-4 Hz), theta (4-8 Hz), alpha (8-12 Hz), and beta (12-30 Hz)—computed for each segment. For instance, the spectral power in the theta band within the 300-600 ms segment may be particularly relevant, as strong P300 responses may exhibit higher energy in the delta-theta range. Variations in relative power across frequency bands may differentiate between healthy and cognitively impaired individuals, with increased theta power and decreased alpha power commonly associated with cognitive decline.

Other frequency-domain features include the dominant frequency or peak frequency in a segment's Fourier spectrum, as well as spectral entropy, which quantifies signal complexity. For very short segments (e.g., 50 ms), time-frequency transforms, such as wavelet decomposition, may be more suitable for capturing evoked oscillations. However, in many cases, band power and wavelet energy at key scales provide sufficient information. Frequency-domain features complement time-domain features by capturing the rhythmic aspects of brain activity—where prolonged cognitive processing may be reflected in stronger theta oscillations, while rapid sensory responses may show increased activity in higher beta frequencies. These insights can be leveraged by the classifier to improve cognitive status assessments.

In some embodiments, ERP-specific features are extracted based on known ERP “landmarks” or prominent time points within a segment. If a segment corresponds to a known ERP component—such as N100 or P300—the system can explicitly measure that component's peak amplitude and latency. Certain prominent peaks, such as Pa (~30 ms, subcortical), P1 (~70 ms), N1 (~100 ms), and P2 (~180 ms), can be identified. For each expected peak, the system can extract its amplitude and latency if it falls within the segment. Additionally, mean amplitude within a narrow time window around the expected peak can be computed to mitigate noise effects.

These targeted ERP features can provide direct quantification of clinically relevant markers. For example, “N1 latency” or “P2 amplitude” can be included as features, allowing the classifier to identify subtle deviations indicative of cognitive impairment. If an individual exhibits an abnormally reduced P2 response, the extracted P2 amplitude feature may be lower, which may serve as an indicator for classification. By incorporating these ERP features, the system can integrate domain knowledge to enhance the interpretability and diagnostic utility of the model.

In some embodiments, the system can automatically detect and log these peaks within each segment using peak detection algorithms, recording their values in the feature vector.

Beyond individual segment features, the system can also derive comparative metrics between segments. For instance, the ratio of amplitudes between two segments—such as early response energy relative to late response energy—may provide insights into cognitive processing patterns. A cognitively impaired profile might exhibit relatively preserved early sensory responses but significantly reduced late cognitive responses, leading to a lower late-to-early ratio compared to a normal profile. Similarly, differences in peak latency between two ERP components, such as N1 and P3, can serve as indicators of cognitive slowing.

Because higher-order features can emerge from relationships between basic segment features, the machine learning algorithm may also implicitly learn these interactions. In some embodiments, the system extracts tens of features per segment, and with multiple segments per AEP epoch, the total number of initial features can be quite large—potentially in the hundreds. While this broad feature set allows for extensive pattern recognition, not all features contribute equally to classification. To refine the dataset, the system can employ a feature selection and optimization process to identify the most informative subset.

Many extracted features correspond to well-established physiological markers. For example, peak amplitudes and latencies of P1, N1, and P2 have known relevance in aging and cognitive decline. Similarly, band-power features align with recognized EEG changes in cognitive impairment, such as increased theta power and reduced alpha power. By extracting these well-established biomarkers, the system ensures that conventional diagnostic indicators can be included while also incorporating novel features—such as segmentation statistics—that may capture subtler patterns not previously identified.

Following feature extraction, each AEP epoch—or an aggregated set of epochs per subject—can be transformed into a high-dimensional feature vector. In one implementation, if a subject's epochs are averaged, a single feature vector may represent their AEP profile. Alternatively, multiple feature vectors can be generated per trial, allowing for ensemble classification or averaged predictions across trials. In some embodiments, the system supports classification at the subject level, meaning a patient can be classified as cognitively impaired or normal based on their AEP data. To enhance robustness, features may be aggregated across all trials per subject, such as averaging feature values across epochs or using the grand-average AEP's characteristics. Ultimately, this feature set can serve as the input for the machine learning classifier, which can determine cognitive status.

### Machine Learning Classification and Model Training

In some embodiments, once features have been extracted from AEP segments, the system can apply ML techniques to classify whether the patterns indicate cognitive impairment. ML allows the system to analyze multiple features simultaneously and uncover complex, non-linear relationships that differentiate impaired from unimpaired brains.

Segmentation facilitates the selection of features used for training machine learning classifiers. These models may be hosted on external servers, with selected features uploaded to an online server for classification into either normal cognitive function or impaired cognitive status.

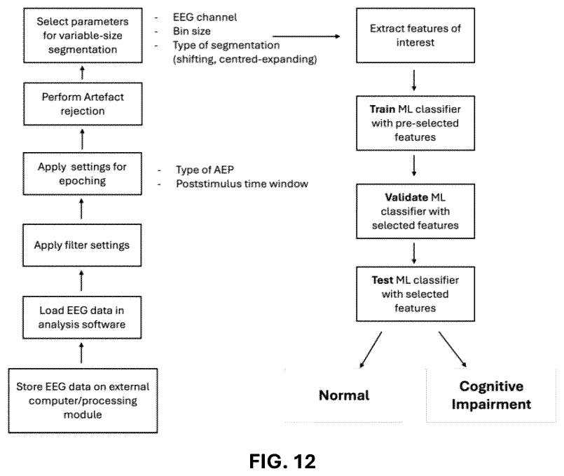

As illustrated in FIG. 12, the classification process involves training a model on labeled data from individuals with known cognitive diagnoses and then using the trained model to predict the cognitive status of new subjects based on their AEP features.

In some embodiments, after cross-validation, the system evaluates the model's accuracy, sensitivity, specificity, and other performance metrics. If the results are unsatisfactory, the system iteratively refines the model. Performance evaluation may include assessing accuracy metrics and generating a confusion matrix to visualize classification outcomes.

If the model does not meet the required performance standards, the user can refine the segmentation of AEP epochs by adjusting segmentation parameters and extracting new features for model training. This iterative process continues until the machine learning classifier achieves a satisfactory level of accuracy.

In some embodiments, segmentation strategies may require adjustment. If an initial segmentation does not capture an essential component, modifications can be made. For instance, if none of the top-ranked features correspond to the 100 ms range—despite research indicating that N100 is a relevant feature—the system can apply a narrower segmentation window (e.g., 80-120 ms) and re-extract features. The segmentation process is adaptable, allowing the user to experiment with different window sizes or overlaps based on feature importance rankings. If multiple top features originate from a specific time range, finer segmentation can be applied in that region; conversely, if a time range lacks meaningful features, segmentation may be too coarse.

In some embodiments, the system supports the addition or removal of feature types to optimize model performance. If spectral features are absent from the top-ranked set, they may be omitted in the next iteration to reduce dimensionality. Conversely, if time-frequency patterns emerge as key differentiators, new features may be introduced.

Once the model is trained and validated to a satisfactory level, it can be finalized and deployed for real-world use. When a new subject undergoes AEP testing, their EEG data is processed, segmented, and analyzed in the same manner as the training data. The extracted feature set is then fed into the trained classifier, which generates a result—such as a probability score of cognitive impairment or a categorical classification (e.g., “impairment detected” vs. “no impairment detected”). The system may also provide a risk score or recommend further evaluation if cognitive impairment is likely.

In some embodiments, because the system is data-driven, it can continuously improve as more data becomes available. In a clinical deployment, as the device is used on additional subjects with confirmed outcomes, new data can be incorporated into the training set to periodically retrain or fine-tune the model. To maintain accuracy and prevent model drift, periodic validation ensures that updates enhance, rather than degrade, performance. If new data suggests that different features become more relevant, the feature selection step is repeated to optimize classification accuracy.

To summarize, the machine learning component transforms the problem into a pattern recognition task: the set of AEP features—including amplitudes, latencies, and spectral characteristics across various segments—forms a multi-dimensional pattern indicative of normal or impaired cognitive function. The classifier learns this distinction from training data, allowing objective, automated cognitive impairment detection. By integrating feature selection and iterative optimization, the system ensures that the final model is both efficient (using a compact set of the most informative features to avoid overfitting) and accurate. This ML-driven approach provides a standardized, reproducible, and scalable method for assessing cognitive function, eliminating the subjectivity associated with traditional ERP analysis.

### Advantages of the Proposed System Over Conventional AEP Analysis

The described system for variable-size segmentation and ML-based analysis of AEPs offers several advantages over traditional AEP and ERP analysis techniques.

Conventional ERP analysis typically focuses on a limited number of components, such as measuring only the P300 peak latency and amplitude. In contrast, the present system examines the entire evoked response across multiple time scales and segments. By employing sequential, shifting, and expanding segmentation techniques, the system captures both early and late components of the AEP, enhancing the likelihood of detecting cognitive impairment regardless of when abnormalities occur in the response.

For example, early sensory changes, such as reduced P2 amplitude around 200 ms, and later cognitive changes, such as a delayed P300 around 350 ms, can both be identified and factored into the assessment. This multi-segment approach mitigates the risk of overlooking abnormalities that fall outside a predefined time window. Essentially, the system provides a comprehensive view of the AEP, whereas conventional methods might analyze only a narrow segment.

By extracting a broad set of features and applying machine learning to weigh their significance, the system can achieve higher sensitivity and specificity in detecting cognitive impairment than single-metric approaches. In traditional analysis, a test might be deemed positive if “P300 latency >X ms” or “P300 amplitude <Y μV.” However, such fixed thresholds can be insensitive, especially for early or mild impairment, where P300 changes may be subtle and subject to variability with age and other factors.

The analysis described here, however, integrates multiple features such as slight P300 prolongation, minor P2 reductions, and increased theta power-to make an assessment. While individual changes might be minor, their combined pattern can provide a more robust indicator that the classifier can recognize. This data-driven fusion of multiple weak indicators results in a stronger overall detection method.

Moreover, the classifier can be adjusted to balance false positives and false negatives, ensuring high specificity (e.g., >90%, so that cognitively normal individuals are rarely misclassified) while maintaining good sensitivity. This makes the system particularly useful for cognitive screening applications.

Another advantage of this system is the removal of subjective interpretation. Traditional ERP analysis often requires experts to manually inspect waveforms, identify peak points, and classify responses as “normal” or “abnormal.” This process introduces potential inconsistencies between evaluators. In contrast, the described system automatically processes EEG data, extracts defined features, and applies a trained ML model to generate an objective result. The model's learned parameters ensure consistent criteria across all subjects.

Additionally, this automation significantly accelerates the analysis process. Results can be generated immediately after data acquisition, eliminating the delay associated with manual review. The objectivity of this approach is particularly valuable in multi-center studies or longitudinal tracking, where consistency over time is essential. Unlike manual interpretation, which may vary based on subjective judgments or waveform irregularities, the ML model evaluates all available data holistically, reducing uncertainty about whether a peak represents noise or a true cognitive response.

In some embodiments, the system's sensitivity to subtle changes in AEPs allows for the detection of cognitive impairment at an earlier stage than conventional methods. For example, while P300 latency prolongation in MCI may be statistically significant in group studies, it might be too subtle to confidently identify in an individual patient using traditional methods. However, the ML system can detect a combination of small changes, such as a 20 ms latency increase along with minor reductions in P2 amplitude or shifts in spectral power, and classify them as indicative of early impairment.

Early detection is critical for conditions such as MCI and Alzheimer's disease (AD), as interventions to slow cognitive decline are most effective when initiated in the earliest stages. By identifying possible impairment before it becomes clinically apparent, the system can provide a proactive tool for preventative healthcare and timely intervention. In effect, it enhances the weak, early “canary in the coal mine” signals in AEPs that might otherwise be lost in variability, making them actionable for clinical decision-making.

The system's use of variable segmentation ensures adaptability to variations in response timing. Traditional fixed-window analyses may miss peaks that occur slightly earlier or later than expected. In contrast, the shifting window approach ensures that if an individual's response is slightly delayed or advanced, at least one segmentation window will capture the peak accurately. Similarly, expanding windows account for prolonged cognitive responses, ensuring that the entire relevant time frame is analyzed.

This adaptability enhances the robustness of the system across individuals with differing brain and hearing profiles. It also allows for effective cognitive assessment across a wide age range. For example, neural response latencies tend to increase with age. A fixed 0-500 ms analysis window might fail to capture late P300 responses in older adults, whereas the system's expanding segmentation, extending up to 700 ms, accommodates such shifts without manual adjustments.

Additionally, because the ML model can be retrained, the system can be customized or calibrated for specific subpopulations. For instance, individuals with hearing loss may exhibit reduced cortical auditory-evoked potentials (CAEPs) due to peripheral hearing deficits rather than cognitive impairment. A specialized model can be trained to include hearing thresholds as additional features or to distinguish cognitive impairment in individuals with baseline hearing loss. This level of personalization is difficult to achieve with conventional one-size-fits-all threshold-based approaches.

The system can also be personalized longitudinally. If a baseline is established while a person is cognitively normal, future tests can be compared against that baseline to detect meaningful deviations over time. The flexibility of the ML framework makes this type of individualized tracking possible.

Furthermore, the system's design supports portable and even wearable implementations. Since it requires only EEG sensors and an auditory stimulus source—both of which can be compact—the entire setup could be incorporated into a headset with integrated earphones and electrodes.

The automated analysis pipeline further enhances efficiency by generating results within minutes of completing an EEG recording. In a busy clinical setting, a 15-minute AEP test followed by immediate analysis could be seamlessly integrated into routine cognitive assessments for at-risk patients.

Moreover, the system's use of advanced signal processing techniques may allow for accurate classification with fewer trials than traditional ERP averaging methods, reducing test duration while maintaining reliability. Classical ERP studies often require 50 or more trials to achieve sufficient signal-to-noise ratios, whereas the ML model in this system can detect impairment on an individual-trial basis or with minimal averaging.

Instead of providing a single numerical output (e.g., “P300 latency=350 ms”), the system offers a more detailed and clinically informative report. For example, it can highlight the specific features that contributed to an impairment classification, such as “Delayed response in the 100-300 ms window and low amplitude in the 300-600 ms window.” This level of insight allows clinicians to better understand a patient's cognitive processing abnormalities and potentially correlate them with specific cognitive domains, such as attention or sensory processing.

In summary, this system leverages modern EEG technology and machine learning to create a highly sensitive, objective, and efficient tool for detecting cognitive impairment. By analyzing auditory-evoked responses with greater depth and precision than conventional methods, it allows for earlier detection of subtle neurophysiological markers of cognitive decline. Additionally, its automation and portability make it suitable for deployment in clinical settings, primary care, and home monitoring-expanding access to cognitive health assessment and facilitating early intervention strategies.

The systems and methods described herein can be applied in various practical scenarios to enhance cognitive health assessment.

The systems and methods described herein can be used to extract AEP features from patients affected by distinct cognitive or mental health disorders, including, but not limited to, Alzheimer's Disease, Psychosis, Depression, Parkinson's Disease, and Schizophrenia.

Memory clinics, neurology offices, and general practitioners can use the system as a rapid screening tool for patients experiencing memory complaints or presenting risk factors for Alzheimer's disease. Since the test is language- and culture-independent, relying solely on auditory stimuli and EEG responses, it is particularly valuable in diverse clinical populations, where traditional cognitive tests may be biased by educational background or linguistic proficiency.

The system's portability allows for at-home cognitive monitoring, allowing users to track their brain function over time. A user with a simple EEG headset (integrated with headphones and connected to a smartphone) can conduct periodic at-home AEP tests, possibly guided by a caregiver or automated app interface. The recorded data is uploaded and processed by the machine learning model, either locally or via cloud-based analysis.

In research and therapeutic settings, the system serves as an objective tool for tracking changes in cognitive function over time. For example, in a clinical trial for a novel drug targeting cognitive improvement in MCI patients, participants can undergo AEP testing at baseline and at scheduled intervals. Improvements in cognitive processing may manifest as partial normalization of their AEP features—such as a shortening of P300 latency or an increase in certain segment amplitudes.

Because the system generates continuous numerical outputs rather than binary pass/fail results, it is sensitive to subtle, progressive changes in cognitive function. This feature also makes it ideal for evaluating cognitive training programs and brain stimulation therapies. If a patient undergoes cognitive rehabilitation, periodic AEP tests can objectively measure whether their neural processing speed or efficiency is improving. For instance, a previously blunted P200 response might increase in amplitude with therapy.

By providing a robust, objective, and scalable biomarker for cognitive function, the system is well-suited for longitudinal monitoring, allowing researchers and clinicians to track therapeutic progress without the limitations of practice effects or patient effort, which often affect traditional cognitive tests. The system offers a tool for measuring intervention efficacy and disease progression in a way that is quantifiable, repeatable, and sensitive to subtle neurophysiological changes.

### Example: Processing and Segmenting EEG Data for Cognitive Analysis

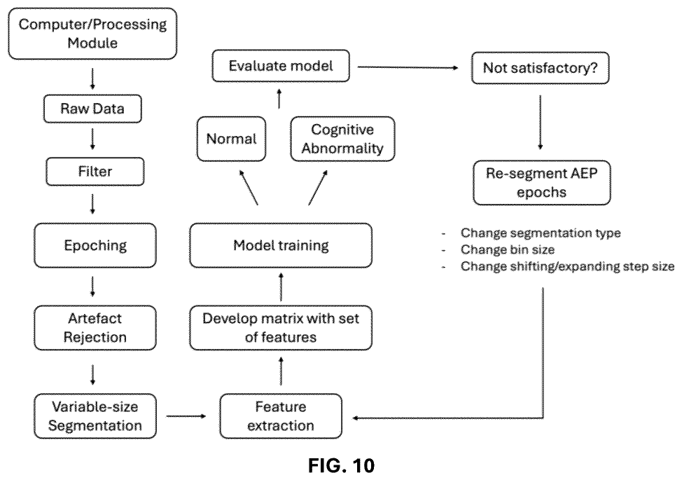

FIG. 10 depicts a flow diagram outlining the process for AEP data acquisition, and machine learning-based classification for cognitive assessment. In some embodiments, the process begins with raw EEG data acquisition, followed by filtering, epoching, artifact rejection, and variable-size segmentation. Extracted features are compiled into a matrix and can be used for training a machine learning model. In some embodiments, the trained model evaluates cognitive status, classifying subjects as either normal or exhibiting cognitive abnormalities. If model performance is unsatisfactory, re-segmentation adjustments-such as modifying segmentation type, bin size, or shifting/expanding step size-can be applied to optimize feature extraction and improve classification accuracy.

In some embodiments, the user first prepares the test subject by placing EEG sensors on the scalp. Insert earphones or overhead headphones are positioned on the subject's ears. During auditory stimulation, the user may instruct the test subject to watch a silent movie. Before starting, the user informs the test subject that the auditory stimulation is about to begin. The user then switches on the EEG amplifier and initiates the delivery of auditory stimuli by pressing the start button on the electronic module.

The recorded EEG trace, containing the pre-amplified, filtered, and digitized signal, is transmitted to an external computer, tablet, or digital processing module. The user opens the analysis software on the external device and loads the raw EEG data file. The dataset includes all EEG channels, along with the trigger codes recorded by the EEG amplifier during auditory stimulation.

In an exemplary embodiment, a master script provides options for either automatic or manual analysis via a user interface, accessible through mouse clicks or touchscreen input.

When manual analysis is selected, the user can specify parameters for each processing step, including segmentation. Once a suitable set of features has been identified, the chosen parameters are saved in a master script labeled “automatic analysis.” This script can automatically extract the desired features and apply them to a machine learning model that demonstrated satisfactory classification performance during training.

To identify optimal features in the AEP signal, the manual analysis process is executed first.

In some embodiments, on the external computer, tablet, or digital processing module, the raw EEG data from all available channels (e.g., 64 channels) is filtered using a low-pass, high-pass, band-pass, or notch filter. Band-pass filters may be applied across different frequency bands, such as 1-3 kHz, 2-10 kHz, or 15 kHz, depending on the analysis requirements.

In some embodiments, the appropriate filter settings are applied by selecting the “Apply Filters” button. The user can apply specific filtering settings to the raw EEG dataset. Pressing the “Filter” button in the analysis software opens a new window where the user can select from low-pass, high-pass, notch, or band-pass filters. If a band-pass filter is selected, the user must input values for both the low-cutoff and high-cutoff frequencies. Once these values are set, pressing the “Apply Filter” button executes the filtering process, updating the raw EEG data accordingly.

Next, the user selects the “Epoching” button, which allows the insertion of trigger codes corresponding to the AEP of interest and the definition of the prestimulus baseline period and epoch length. By selecting the “Extract Epochs” button in the Epoching analysis window, the user extracts the epochs for the respective AEP. The trigger selection is necessary because the raw EEG data file may contain multiple triggers corresponding to different auditory stimuli. For example, in a traditional oddball paradigm, standard tones may be assigned trigger code 1, while deviant tones may be assigned trigger code 2. Consequently, the raw EEG dataset will contain both trigger codes corresponding to the standard and deviant tones. The user selects the trigger corresponding to the AEP of interest and defines the time window (post-stimulus period) and the pre-stimulus baseline period. Pressing the “Perform Epoching” button executes the script, extracting the epochs associated with the selected trigger.

Artifact rejection can be performed by pressing the dedicated button in the analysis software and selecting one of the available artifact rejection methods. The extracted epochs may contain non-auditory-related brain activity, such as artifacts from muscle activity, eye blinks, or power line noise. By pressing the “Artifact Rejection” button, the user opens a window with options to select different artifact rejection algorithms, such as “Simple Voltage Threshold” or “Independent Component Analysis” (ICA). Upon selecting the desired method, the software screens the extracted epochs. Any epochs flagged due to potential artifacts are removed from the dataset. The cleaned AEP dataset is then saved to the local hard drive of the external computer, tablet, or digital processor.

In some embodiments, the cleaned epochs are saved in a structured format (e.g., column or row). In a column format, the first column contains the sample number, the second column records the time in milliseconds, and the third column stores the amplitude value (typically in microvolts) of the AEP.

The saved epoch dataset includes all individual epochs for a given auditory stimulus type. For example, if stimulus type 1 was presented 100 times, the EEG amplifier records 100 corresponding trigger codes. Selecting the correct trigger should result in extracting 100 AEP epochs. The dataset includes both the individual epochs and their averaged waveform, stored in a structured format on the computer, tablet, or digital processing module.

The user can then segment the EEG dataset into variable-sized segments by selecting the dedicated button in the analysis software, which opens a window for inputting segmentation parameters. The user then defines the parameters for segmentation, including: EEG channels to be analyzed; segmentation type (sequential, shifting, or centered-expanding); bin size (length of each segment); expanding step size (for centered-expanding segmentation); and shifting step size (for shifting segmentation). Selecting “Sequential Segmentation” requires the user to specify only the bin size, while “Shifting Segmentation” also requires a shifting step size. For “Centered-Expanding Segmentation,” the user must input the expanding step size. Once the parameters are set, pressing the “Perform Variable-Size Segmentation” button executes the process. Depending on the selected parameters, various segments of the AEP epoch are extracted and stored for further analysis. Variable-size segmentation can be applied to all EEG channels or limited to specific brain regions (e.g., temporal or frontal electrodes).

Sequential segmentation starts at time point 0 (stimulus onset) and divides the AEP into fixed-length segments. For example, with a bin size of 5 ms and a post-stimulus period of 500 ms (sampling rate 1000 Hz, 500 samples), the segmentation extracts: segment 1:0-5 ms; segment 2:5-10 ms; segment 3:10-15 ms; . . . and so on until 500 ms.

Shifting segmentation applies overlapping segments that shift incrementally across the AEP epoch. For example, if the bin size is 5 ms and the shifting step size is 2 ms, the segments extracted would be: segment 1:0-5 ms; segment 2:2-7 ms; segment 3:4-9 ms; . . . and so on until the 500 ms mark.

Centered-expanding segmentation centers the segmentation at a specific time point and expands outward with increasing segment lengths. For example, if centered at 250 ms, with an initial bin size of 5 ms and an expanding step size of 5 ms, the segments extracted would be: segment 1:247.5-252.5 ms; segment 2:242.5-257.5 ms; segment 3:237.5-262.5 ms; . . . and so on until reaching the end of the AEP epoch.

These segmentation techniques allow for a detailed analysis of short, middle, and long-latency auditory-evoked responses, significantly enhancing the utility of AEPs as potential biomarkers for cognitive impairment.

Each segmentation process generates a dataset with segments labeled 1-X, based on the selected parameters. The dataset includes: segment number; value index; time (ms); and extracted amplitude values. Each segment can contain one or multiple values, indexed numerically. The corresponding time value of each sample is also recorded. These structured datasets are saved for each EEG channel selected for analysis.

Each segment may contain a single or multiple values, which can be used to extract new features. To compute the average amplitude within a given segment, the values within that segment (e.g., S1-V1, S1-V2, S1-V3, S1-V4, and S1-V5) are averaged. This computed metric may be assigned as F_S1_AVERAGE_IND if extracted from an individual AEP epoch or F1_S1_AVERAGE_AV if derived from an averaged AEP epoch.

Similarly, a new feature may be extracted to represent the local minimum value within a specific segment. For example, if S2-V1 is the lowest value in segment 2, the extracted feature is labeled F_S2_MIN.

In some embodiments, the segmentation step may be iterated multiple times, allowing for different segmentation techniques and parameters to be applied to the AEP epochs. The output from each segmentation process can be saved in a folder named according to the selected parameters. For example: Folder Y: Seq_BS5_AEP2_0_500 contains the dataset from sequential segmentation with a bin size of 5 ms, applied to AEP 2, where the start and end of the auditory-evoked potential time window were 0 ms and 500 ms. Folder X: Exp_BS10_C100_AEP3_0_500 contains the dataset from expanding segmentation with a bin size of 10 ms, centered at 100 ms, applied to AEP 3, where the start and end time were 0 ms and 500 ms.

Each of these folders contains extracted segments and their corresponding values from all EEG channels selected for analysis. In some embodiments, the high number of generated AEP segments allows for the extraction of diverse features, which may be used for training machine learning classifiers. A dedicated script loops through each extracted segment and computes specific features such as: mean amplitude of a segment; slope of a given set of values (e.g., first two values of segment 1); and variance, standard deviation, and frequency-domain features.

The segmented dataset is saved to the external computer, providing access to a variety of distinct segments from which multiple features can be extracted.

To extract features from each segment, the user runs dedicated scripts that compute various metrics such as average amplitude, slope, variance, Fast Fourier Transform (FFT), local minimum, and local maximum.

Extracted features are stored in row, column, or matrix format for further processing. Machine learning models can be trained on this feature set, and model performance is iteratively evaluated. If performance is unsatisfactory, the AEP epochs can be re-segmented with adjusted parameters, producing a new set of unique segments. The new feature set is extracted and used to retrain the machine learning model, repeating the process until an optimal feature set is identified that leads to high classification accuracy.

The software responsible for processing the raw EEG data into variable-sized segments consists of multiple scripts dedicated to each processing step.

The software architecture on the computer, tablet, or processing module consists of multiple scripts, each dedicated to a specific processing step.

The automatic analysis script sequentially executes scripts 1-6, below, using pre-set parameters to extract relevant features from AEPs. These features achieve satisfactory classification performance when training machine learning models. The script directly outputs the extracted AEP features from the test subject, which are then classified by a machine learning algorithm to determine whether they indicate cognitive impairment or normal cognitive function The analysis can also be performed manually by running each of the scripts individually.
Script 1: Load EEG Data. This script loads the raw EEG data stored on the computer, tablet, or processing module into the analysis software. All numerical values from all EEG channels are imported, and the EEG waveforms from each channel are displayed on the computer screen.
Script 2: Filtering. The software applies filter settings chosen by the user, removing all frequency components outside the selected range for each EEG channel. The numerical values are updated accordingly, and the filtered EEG waveforms are displayed.
Script 3: Epoching. The EEG data is segmented based on the trigger codes corresponding to auditory stimuli. The software scans the EEG traces for these trigger codes and extracts the relevant AEP epochs. The user can specify the epoch window and prestimulus baseline period. The software then sets time 0 at (or before) the stimulus onset and extracts numerical values from 0 to the end of the defined epoch (e.g., 500 ms), including the prestimulus period.
Script 4: Artifact Rejection. This script applies artifact rejection algorithms to remove noisy AEP epochs. The software may use a simple voltage threshold to flag and remove epochs that exceed a predefined range or employ more advanced methods such as independent component analysis (ICA) to detect and eliminate ocular or muscle-related noise.
Script 5: Variable-Size Segmentation. The software performs variable-size segmentation on the cleaned AEP epochs, as described earlier, based on user-defined parameters.
Script 6: Feature Extraction. This script extracts specific features from the segmented AEP data. It applies functions to calculate metrics such as average amplitude, variance, standard deviation, local minima, and maxima. The script iterates through all extracted segments and stores the computed features in a structured format (row, column, or matrix).

Before closing the analysis software, all user-specified parameters are saved in a structured format (table, matrix, or list). This allows for reproducible analysis and allows for the automatic extraction of the same AEP features for subsequent test subjects to validate and apply the trained machine learning model.

The elements of the figures are not exclusive. Other embodiments may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed.

The following specific embodiments are not exclusive nor exhaustive but listed as specific uses presented herein.

Use of speech syllables with aim to test speech processing by alternating a standard speech syllable with one out of multiple different deviant speech syllables, with the aim to assess brain responses sensitive to subtle changes in speech fundamental frequency or harmonics, duration or spatial location (predominantly presented at left or right ear through manipulation of interaural time difference).
Use of pure tones presented at 500 ms ISIs or ISIs lower than 200 ms for fast auditory stimulus presentation.
Use of deviant stimuli at fixed stimulus presentation rates (for example, every 6th stimuli is a deviant stimulus) while remaining tones are standard stimuli.
Use of AEP segments derived from simple oddball paradigms or more complex multi-feature MMN paradigms with the aim to develop high level matrices of AEP segments and features representing neuronal changes to speech, frequency, duration, presence of absence of a stimulus (omission) or auditory spatial location.
Use of AEPs with variable sizes derived from paired click or paired chirp paradigms, with varying interchirp intervals or intertrain intervals.
Use of AEPs derived from a combination of stimulus sequences using pure tones or speech syllables.
Use of features extracted from different segments, derived from different AEPs presented in the same stimulus sequence.
Use of features extracted from different segments, derived from different AEPs presented in the same stimulus sequence measured at one or multiple electrodes, predominantly located at one scalp site (for example frontal or temporal) or a combination of electrodes at different scalp sites.
Use of features from different segments, derived from different AEPs presented in different stimulus sequences.
Use of features extracted from different AEP segments, derived from different AEPs presented in stimulus sequences aimed to test the same cognitive function (attention, working memory, time perception).
Use of features extracted from different AEP segments, derived from different AEPs presented in stimulus sequences aimed to test the different cognitive function (attention, working memory, time perception).
Use of features extracted from different deviant AEP segments, normalized to features from a standard AEP segment. For example, mean amplitude in deviant AEP segment subtracted or divided by mean amplitude in standard AEP segment.
Use of AEP segments with variable sizes, extracted from the same auditory stimulus paradigm.
Use of AEP segments with variable sizes, extracted from different auditory stimulus paradigms.

### EXAMPLES

### Example 1: Segmentation of AEPs in Patients with Attention Deficit Hyperactivity Disorder (ADHD)

The following dataset has been collected in healthy volunteers (n=30) and participants who received a formal diagnosis with attention/deficit hyperactivity disorder (ADHD, n=27). The EEG data was recorded with a sampling rate of 2.5 kHz using an actiCHamp EEG system from Brain Products. An Etymotic ER3C insert earphone was used to deliver the acoustic stimuli. While listening to auditory stimuli, participants were asked to watch a silent movie without subtitles.

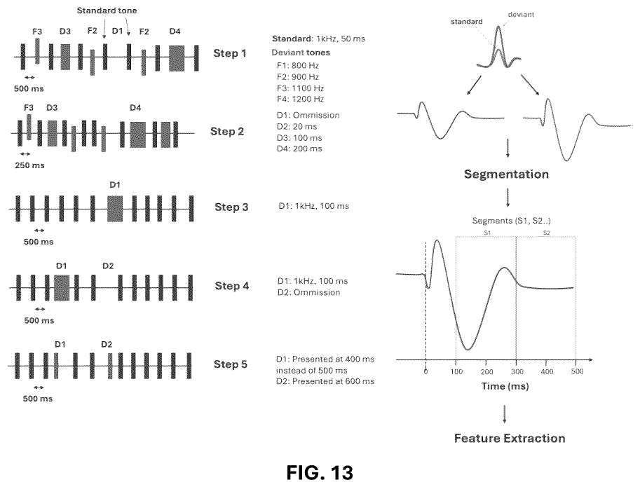

Five different auditory stimulation paradigms were used (steps) including different deviant stimuli differing in duration, frequency or varying interstimulus intervals (FIG. 13). The standard tone consisted in each of the paradigms of a 1 kHz tone with a duration of 50 ms. Five different acoustic paradigms (steps 1-5) were used to extract a variety of AEPs. Steps 1-2 were multifeatured MMN paradigms, using the mentioned standard stimulus and 8 different deviant stimuli that either differed from the standard tone in duration or frequency. An additional deviant tone involved a complete omission (absence of any stimulus). Step 3 used a single deviant of 1 kHz tone with a duration of 100 ms. Step 4 included a deviant tone of 100 ms and an omission. Step 5 deployed deviants that varied in the interstimulus interval by being presented at 400 or 600 ms instead of 500 ms to assess timing variability. Each step had a duration of approx. 8-12 minutes. The experiment involved the presentation of different acoustic stimuli, including multi-feature Mismatch Negativity Paradigms for the extraction of multiple auditory-evoked potentials.

### Processing of Collected EEG Datasets

Continuous EEG was imported from Brain Vision files into EEGLAB (v2021.1) in the Matlab environment, loading channels 1-63. To prevent aliasing prior to resampling, the data were low-pass filtered at 500 Hz and then downsampled to 1000 Hz. Standard 10-05 electrode coordinates were assigned using the EEGLAB/DIPFIT standard template (standard_1005.elc). The resampled data were subsequently band-pass filtered from 1 to 30 Hz (1 Hz high-pass and 30 Hz low-pass) to remove slow drifts and high-frequency noise. Finally, the data were re-referenced to the mastoid electrodes (TP9 and TP10; corresponding to M1/M2), after which the preprocessed continuous dataset was inspected using a custom EEG review GUI for manual quality control and further processing steps (epoching and artifact handling) performed downstream. Bad channels were interpolated.

### Extract Epochs

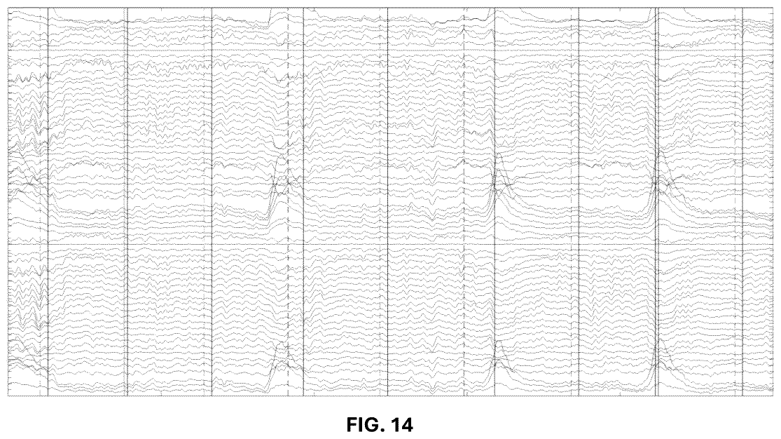

Preprocessed EEGLAB.mat file were loaded in a second step. The trigger codes were saved in a separate binlister file and were imported for each step to extract the epochs with AEPs. Data were epoched using user-defined time limits and baseline-corrected to the pre-stimulus period. Trials were automatically marked for artifacts using a simple voltage threshold (+100 μV) across channels 1-61 within the epoch window; the artifact flags were exported and used to remove the marked epochs. Finally, for each remaining trigger/bin label in the dataset, we generated a trigger-specific epoched dataset and exported both the single-trial epochs and the average ERP waveform to CSV files. FIG. 14 shows an example of an EEG trace where the triggers corresponding to each stimulus are marked in the trace.

In each paradigm, the standard and deviant AEPs were extracted with time windows of −100-500 ms for steps 1, 3, 4 and 5 and −50-250 ms for step 2. In step 1 and step 2, each deviant stimulus was presented 80 times each, while the standard stimulus was presented 8×80×2=1280 in addition to 15 presentation at the beginning to prime the standard stimulus. The total duration of this step was approx. 16 min in step 1, and approx. 8 min in step 2. The individual extracted epochs of each deviant and standard stimulus in each step are saved in an excel file. A separate excel file with the numerical values of the averaged EEG trace of each deviant is saved as well.

Rather than focusing on a specific auditory-evoked potential such as the MMN, P300 or P50 component, we used an entire segment from 100-300 ms and extracted multiple features including minimum/maximum amplitudes and latencies. This underlies the assumption that composite markers of AEPs include various metrics of the AEP waveform rather than a single metric (such as MMN amplitude, or P300 latency) are more suitable features for ML models and can therefore maximize the potential of AEPs in the classification of healthy and cognitive impairment.

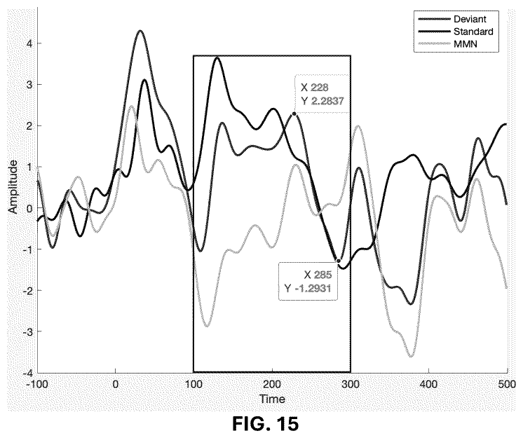

A separate script has been used to extract the minimum and maximum values in a specified time window (here 100-300 ms, FIG. 15) in each averaged AEP. The following parameters were extracted from each deviant waveform: (1) Minimum amplitude, (2) Latency of minimum amplitude, (3) Maximum amplitude, and (4) Latency of maximum amplitude. This corresponds to the following values in FIG. 15: (1) Minimum amplitude: −1.2931 μV, (2) Latency of minimum amplitude: 285 ms, (3) Maximum amplitude: 2.2836 uV, (4) Latency of maximum amplitude: 228 ms. A matlab script was used to extract the Max Value, TimeAtMax, Min Value, TimeAtMin in the 100-300 ms value of the averaged AEPs.

### ML Analysis on Averaged AEPs

EEG-derived middle-latency features were extracted from a predefined subset of frontal electrodes (Fp1, Fp2, AF7, AF3, AFz, AF4, AF8) and four auditory triggers (S152, S158, S200, S210). From the original dataset (57 participants, 2212 columns), 112 features matching the specified window, electrodes, and triggers were selected. Data were split into training and test sets using a stratified 80/20 split. A machine-learning pipeline consisting of standardization, principal component analysis (PCA), and a linear support vector machine (SVM) was implemented. Hyperparameters for the number of PCA components (fixed values and variance-based thresholds) and the SVM regularization parameter C were optimized using 5-fold cross-validation on the training set. Model performance was assessed using cross-validated accuracy during training and accuracy, sensitivity, and specificity on the held-out test set. To aid interpretation, SVM weights were back-projected from PCA space into the original feature space to identify features contributing most strongly to the classification decision.

The optimal model used PCA retaining 90% of the variance (15 components) combined with a linear SVM (C=10), achieving a mean cross-validated accuracy of 80.0% (±8.3%). On the independent test set, the model reached an accuracy of 91.7%, with perfect sensitivity for the diseased group (100%) and a specificity of 83.3% for controls. The confusion matrix showed no false negatives and a single false positive, indicating robust detection of diseased participants in this split. The most influential features were predominantly middle-latency timing and amplitude measures from frontal electrodes, particularly latency-to-maximum and latency-to-minimum features associated with triggers S152 and S158. Overall, this configuration demonstrated the strongest balance between cross-validated performance and test-set generalization among the evaluated models.

### Advanced ML Analysis Using Segmented Single Trials AEPs

EEG auditory-evoked potential (AEP) data were imported in Python and organized by participant and stimulus condition (tones S152, S158, S200, S210). Metadata and signal arrays were checked for internal consistency (e.g., expected dimensions, presence of required tones, and alignment of time vectors) to ensure uniform formatting across participants prior to feature generation.

For each stimulus condition, signals were handled in a broad peri-stimulus epoch of −100 to +500 ms relative to stimulus onset. A pre-stimulus baseline window (−100 to 0 ms) was extracted for every trial and channel, and the mean baseline level was computed. Signals were then baseline-corrected on a trial-by-trial and channel-by-channel basis by subtracting each trial's baseline mean from its post-stimulus waveform.

To reduce single-trial variability and standardize the number of observations per condition, trials were aggregated into non-overlapping blocks of 20 trials (parameter nrep=20). For each tone, consecutive groups of 20 trials were averaged to form multiple “repetition” waveforms per participant (equal to floor (N_trials/20)), and incomplete trailing blocks were discarded (drop_incomplete=True). Feature extraction focused specifically on the late-latency AEP segment from 100 to 300 ms post stimulus. Latencies were computed relative to the start of this analysed window; thus, latencies can be converted to post-stimulus units by adding +100 ms.

From each averaged repetition waveform, channel-wise morphological features were computed within the 100-300 ms window, including peak amplitude and peak latency, trough amplitude and trough latency, peak-to-peak amplitude, rise slope (peak amplitude divided by peak latency), fall slope (absolute trough amplitude divided by trough latency), and positive and negative area (time integral of the positive and negative portions of the waveform). In addition, global features were derived from Global Field Power (GFP) across channels, including mean GFP, maximum GFP, and the latency of maximum GFP. All features were assembled into a tabular format for downstream modelling.

Feature tables were reshaped into a wide “features× sample” matrix in which tone- and channel-specific features were grouped into a single feature set per sample. Samples lacking a complete set of required tone-derived features were excluded (i.e., rows with missing tone data were removed), yielding a consistent feature representation across participants and repetitions.

The dataset was divided into training and held-out test subsets using a stratified split. As a screening step, models were trained using one channel at a time and one tone at a time to compare discriminative performance across electrode locations and stimulus conditions. Hyperparameters and feature configurations were optimized via cross-validation/grid search, including the number of PCA components and combinations of electrodes and tones. Multiple classifier families were compared using optimized settings, including Random Forest, RBF-kernel SVM, linear SVM, logistic regression, and XGBoost. Performance was summarized using ROC-AUC, accuracy, and confusion matrices on the held-out test set.

Across the evaluated models, the strongest held-out performance was observed for a Random Forest classifier (test ROC−AUC=0.909, test accuracy=0.800, sensitivity for the positive class≈0.889, specificity for the negative class≈0.727). In the reported grid-search results, the best-performing linear model was logistic regression with PCA (5 components, C=1), achieving test ROC−AUC=0.904 and test accuracy=0.825.

### Example 2: Segmentation of AEPs in Patients with Alzheimer's Disease

Additional pilot work in participants with Alzheimer's Disease (AD) and healthy volunteers, using different auditory stimulus paradigms, indicate potential differences in amplitudes for specific segments of the AEP (n=3 for AD, n=4 for healthy volunteers). The data suggests that distinct segments of the AEPs, derived from distinct stimulus paradigms rather than from a single traditional oddball paradigm, may be useful in the diagnosis of Alzheimer's Disease.

The AD pilot involves an optimized multi-feature AEP battery specifically designed to test a variety of cognitive functions including speech processing, sensory inhibition (gating), auditory memory, auditory spatial processing and duration discrimination, with the aim of identifying early AD-related dysfunction.

Auditory memory (Step 6) was tested by using a standard tone of 700 Hz with a duration of 75 ms, and deviant tones of the same frequency with a duration of 25 ms. An interstimulus interval of approx. 167 ms was used to assess neuronal responses in a very fast paradigm. Deviant responses were presented at 1 Hz, standard tones at 6 Hz, hence, every 6th tone was a deviant tone while the remaining stimuli were standard tones.

To assess speech processing (Step 8), standard vowels were used with a duration of 170 ms including 10 ms rise/fall times. Formant 0, 2 and 3 of all vowels remained identical at 100 Hz, 1090 Hz and 2350 Hz. Standard vowels have a Formant 1 frequency of 580 Hz and are 100 ms in duration. During the priming phase, the standard vowel is presented 15 times. Next, the standard and deviant vowels are presented each with a probability of 0.5. Four different deviant categories are used: vowels with a different F1 frequency (Dfl, 430 Hz, or 730 Hz), vowels with a shorter duration (Ddur, 85 ms instead of 170 ms), vowels with a 20 ms silent gap in the middle (Dsil) and vowels presented with an interaural time difference (vowel is presented 800 ums earlier to the right or left ear) to simulate a deviance in sound location (Dloc). After each standard vowel, a deviant vowel of each deviant category is presented (S—Dfl—S—Dloc—S—Dsil—S Dloc—Ddur—S— . . . ), deviants of the same category are never presented consecutively. Interstimulus interval is fixed at 500 ms. Duration: ~8 min.

To assess recovery from prior stimulation (Step 4), a paired chirp paradigm was used in which a pair of chirps were presented at an interval of 500 ms. The intertrain interval was 3500-5000 ms.

### Data Processing

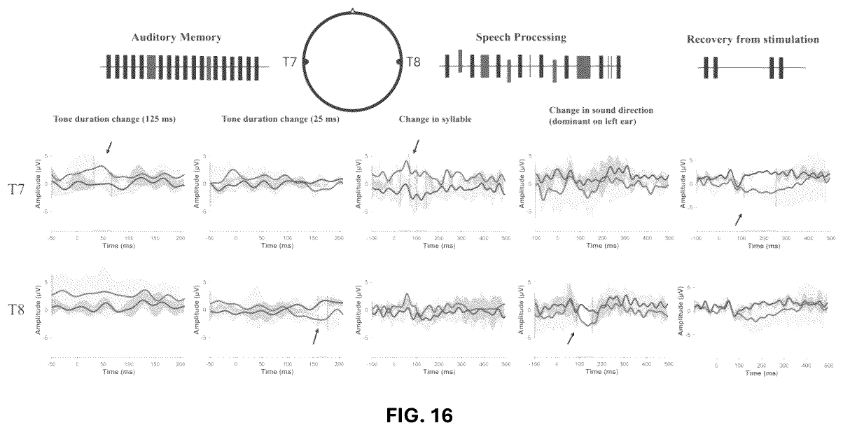

The MATLAB batch script loads one or more BrainVision EEG recordings into EEGLAB, assigns standard 10-05 channel locations. The continuous data was then resampled to 4000 Hz and subjected to line-noise suppression by filtering out line noise through spectrum interpolation. The data was resampled to 250 Hz for long latency responses and was re-referenced to the Nose electrode. Results show altered AEP amplitudes in AD patients (grey line, n=3) at temporal lobe sites (FIG. 16).

This data suggests that segments of the AEP, derived from distinct auditory stimulus paradigms (aimed at testing different cognitive functions) may be used to detect cognitive impairment associated with Alzheimer's Disease. By measuring different AEPs associated with a range of cognitive functions, key AEP segments can be identified which may be predictive of Alzheimer's Disease prior to the onset of clinical symptoms, for example, at the stage of mild cognitive impairments.

We claim:

1. A system for segmenting auditory-evoked potentials (AEPs) from electroencephalography (EEG) signals, the system comprising:
an auditory stimulator configured to present controlled auditory stimuli to a subject;
an EEG acquisition device including a plurality of sensors adapted to detect electrical brain signals from a scalp of the subject in response to the auditory stimuli; and
a data processing unit operatively coupled to the EEG acquisition device, the data processing unit configured to:
receive digitized EEG signals time-locked to the presented auditory stimuli via trigger information;
extract from the digitized EEG signals a series of time-locked epochs, each epoch corresponding to at least one auditory stimulus;
subdivide each epoch into a plurality of segments for subsequent feature analysis using a segmentation process to produce segmented data; and
store the segmented data in a structured format for subsequent feature extraction and analysis.

2. The system of claim 1, wherein the segmentation process comprises:
selecting user-defined segmentation parameters, wherein the segmentation parameters include at least one segmentation mode; and
generating segments from each epoch according to the at least one segmentation mode.

3. The system of claim 2, wherein the segmentation mode is selected from a group comprising sequential segmentation, shifting segmentation, expanding segmentation, or a combination thereof.

4. The system of claim 2, wherein the segmentation parameters further include a bin size, a shifting step size, an expanding step size, and/or a segmentation center point.

5. The system of claim 4, wherein:
in sequential segmentation, the epoch is divided into non-overlapping segments of fixed or variable lengths that collectively cover a post-stimulus time window;
in shifting segmentation, a sliding window of user-defined fixed length moves across the epoch in overlapping increments defined by the shifting step size; and
in expanding segmentation, segments are generated that cumulatively extend outward from a predetermined central time point by increments defined by the expanding step size.

6. The system of claim 1, wherein the auditory stimulator comprises a digital signal processor (DSP) configured to transmit a synchronization signal or trigger code to the EEG acquisition device, thereby marking an onset time of each auditory stimulus.

7. The system of claim 1, wherein the EEG acquisition device includes an analog-to-digital converter and low-noise amplifiers configured to filter and digitize the electrical brain signals at a sampling rate of at least 500 Hz.

8. The system of claim 1, wherein the data processing unit is further configured to apply one or more filters to the digitized EEG signals prior to epoch extraction, the filters comprising at least one of:
a band-pass filter to isolate a frequency range containing cognitive event-related responses; or
a notch filter to remove line-noise interference.

9. The system of claim 1, wherein the data processing unit is configured to remove artifacts from each epoch based on at least one criterion selected from the group consisting of: a peak-to-peak amplitude threshold, a flatline detection threshold, and an independent component analysis (ICA) approach to remove ocular or muscle artifacts.

10. The system of claim 2, wherein the data processing unit is further configured to iteratively adjust the segmentation parameters based on classification accuracy feedback from a machine-learning module, so as to automatically determine an optimal set of segmentation parameters for feature extraction.

11. A method of analyzing EEG signals associated with auditory stimuli to identify features of auditory-evoked potentials (AEPs), the method comprising the steps of:
presenting one or more auditory stimuli to a subject while recording EEG signals;
associating each auditory stimulus with a trigger code indicative of a stimulus onset;
extracting from the recorded EEG signals a plurality of time-locked epochs, each epoch corresponding to at least one auditory stimulus;
segmenting each epoch into a plurality of segments; and
extracting features from each segment for subsequent analysis.

12. The method of claim 11, wherein the step of segmenting each epoch further comprises:
receiving user-defined segmentation parameters including at least one segmentation mode; and
generating segments according to the at least one segmentation mode.

13. The method of claim 12, wherein the segmentation mode is selected from a group comprising sequential segmentation, shifting segmentation, expanding segmentation, or a combination thereof.

14. The method of claim 12, wherein the segmentation parameters further comprise a bin size, a shifting step size, an expanding step size, and/or a segmentation center point.

15. The method of claim 13, wherein sequential segmentation divides the epoch into non-overlapping segments, shifting segmentation generates overlapping segments by moving a fixed-length window in increments, and expanding segmentation produces cumulative segments expanding from a central time point.

16. The method of claim 12, wherein the step of segmenting each epoch into a plurality of segments further comprises iteratively performing segmentation with varying parameter values and selecting segmentation parameters based on an evaluation of classification performance from a machine-learning model trained on features extracted from the segments.

17. The method of claim 11, further comprising a step of extracting one or more features from each segment, wherein the features include at least one of:
average amplitude, minimum amplitude, maximum amplitude, or peak-to-peak amplitude range within the segment;
slope or rate of change in voltage over time within the segment;
latency measurements including the time of a maximum or minimum deflection relative to the stimulus onset;
area under an amplitude-versus-time curve indicating total evoked response;
frequency-domain metrics derived via Fourier or wavelet transforms, including band power (delta, theta, alpha, beta) or spectral entropy; and
event-related potential component metrics if present in the segment.

18. The method of claim 11, wherein the recording of EEG signals in step (a) is preceded by a filtering step, the filtering step comprising at least one of:
applying a band-pass filter to reduce slow drifts and high-frequency noise;
applying a notch filter to remove line noise; and
down-sampling the filtered signals to a lower sampling rate after recording to facilitate subsequent segmentation.

19. The method of claim 11, further comprising:
training a machine-learning model using the features extracted from each segmented epoch to classify whether the subject exhibits a cognitive impairment.

20. A non-transitory computer-readable medium storing instructions which, when executed by one or more processors, cause the processors to perform a method for segmenting and structuring auditory-evoked potentials (AEPs) from EEG data, the method comprising:
receiving raw EEG data recorded while a subject is presented with at least one auditory stimulus;
identifying trigger codes in the raw EEG data to demarcate stimulus onset times;
extracting, based on the trigger codes, a set of time-locked epochs; and
subdividing each epoch into a plurality of segments according to configurable segmentation parameters.

21. The non-transitory computer-readable medium of claim 20, wherein the segmentation parameters include at least one segmentation mode.

22. The non-transitory computer-readable medium of claim 21, wherein the segmentation mode is selected from a group comprising sequential segmentation, shifting segmentation, expanding segmentation, or a combination thereof.

23. The non-transitory computer-readable medium of claim 21, wherein the segmentation parameters further comprise a bin size, a shifting step size, an expanding step size, and a segmentation center point.

24. The non-transitory computer-readable medium of claim 22, wherein the segments are generated as:
non-overlapping segments in a sequential mode;
overlapping segments via a sliding window in a shifting mode; or
cumulatively expanding segments in an expanding mode.

25. The non-transitory computer-readable medium of claim 20, wherein the instructions further cause the processors to execute at least one artifact rejection routine that removes epochs containing artifacts exceeding a predefined threshold prior to segmentation.

26. The non-transitory computer-readable medium of claim 20, wherein the instructions further cause the processors to apply at least one filtering operation to the raw EEG data prior to epoch extraction, the filtering operation comprising a band-pass filter, a high-pass filter, a low-pass filter, or a notch filter.

27. The non-transitory computer-readable medium of claim 20, wherein the instructions further cause the processors to store data from the plurality of segments in a structured arrangement that associates each segment with at least one time index, amplitude value, or frequency-domain representation.

28. The non-transitory computer-readable medium of claim 20, wherein the instructions optionally provide for computing one or more segment-specific metrics, including amplitude statistics, slope measurements, or spectral power estimates, and outputting said metrics for display or subsequent analysis.
